(19) United States
(12) Patent Application Publication
(10) Pub. No.: US 2009/0305381 A1
(43) Pub. Date: Dec. 10, 2009

(54) Activated Polymers Binding Biological Molecules

(76) Inventors: Marcela Bilek, New South Wales (AU); David McKenzie, New South Wales (AU); Niel Nosworthy, New South Wales (AU); Aleksey Kondyurin, New South Wales (AU)

Correspondence Address: AMSTER, ROTHSTEIN & EBENSTEIN LLP, 90 PARK AVENUE, NEW YORK, NY, 10016, US

(21) Appl. No.: 12/225,022

(22) PCT Filed: Mar. 15, 2007
(86) PCT No.: PCT/AU2007/000321

§ 371 Date: Jul. 29, 2009

(30) Foreign Application Priority Data
Mar. 15, 2006 (AU) 2006901344

(51) Int. Cl. C12N 11/08; C08F 110/02; C08L 89/00; C08G 63/91

(52) U.S. Cl. 435/180; 526/352; 525/54.1; 525/54.2

(57) ABSTRACT

The present invention relates to activated polymer substrates capable of binding functional biological molecules, to polymer substrates comprising bound and functional biological molecules, to devices comprising such substrates and to methods of producing them.

### FIELD OF THE INVENTION

The present invention relates in particular, but not exclusively, to activated polymer substrates capable of binding functional biological molecules, to polymer substrates comprising bound and functional biological molecules, to devices comprising such substrates and to methods of producing them.

### BACKGROUND OF THE INVENTION

The advent of diagnostic array technology (where for example protein, antibody or other biological molecule/s is/are attached at discrete locations on a polymer surface to allow attachment of other molecules of interest (target molecules) and where means is provided of detecting the attachment of the target molecules) has led to an increased demand for surfaces capable of binding to biological molecules such as antibodies, other proteins and nucleic acids. It is similarly necessary in other applications, such as for example biosensors, medical devices where biocompatible surfaces are required and in the screening of active agents against drug targets, that surfaces capable of binding to biological molecules are required.

An ideal surface for these applications should bind proteins or other biological molecules while preserving their functionality. The binding needs to be strong and stable over extended periods to allow repeated washing steps during processing. Many present arrays are based on a 96 well polymer microtitre plate. In many of these technologies the protein binding to the polymer surface is attached through non-specific physisorption, leading to losses of protein during washing and variability in the degree of attachment given that the attachment process is molecular species dependent. Functionality of physisorbed proteins depends strongly on the energetics of the interaction with the surface and will vary across proteins.

A number of groups have conducted work in relation to use of plasma gas treatments of polymer surfaces in order to attach biological molecules. Generally, however, techniques such as those disclosed by Wu et al (U.S. Pat. No. 5,922,161), Gsell et al (U.S. Pat. No. 5,258,127), Rosier et al (US patent publication no. 2004/0112518), Hsu et al (U.S. Pat. No. 5,306,768) and Zamora et al (US patent publication no. 2002/0009604) are associated with one or more of the problems that functionality of the biological molecule is not shown to be retained and additional physical and/or chemical treatment steps are required. Additionally, the surfaces are often subject to rapid hydrophobic recovery such that surface properties change over time.

The present inventors have demonstrated that by exposing a polymer surface to plasma treatment under plasma immersion ion implantation (PIII) conditions it is possible to secure strong binding of a range of biological molecules to the treated polymer surface, to minimise and/or delay hydrophobic recovery of the surface and to thereby maintain functionality of the bound biological molecule.

It is with the above background in mind that the present invention has been conceived.

### SUMMARY OF THE INVENTION

According to one embodiment of the present invention there is provided an activated polymer substrate capable of binding a functional biological molecule, the substrate comprising a hydrophilic surface activated to enable binding to said biological molecule and a sub-surface comprising a plurality of cross-linked regions.

According to another embodiment of the present invention there is provided a polymer substrate functionalised with a functional biological molecule, the functionalised polymer substrate comprising a hydrophilic surface with the biological molecule bound thereto and a sub-surface comprising a plurality of cross-linked regions.

According to another embodiment of the present invention there is provided a device comprising an activated polymer substrate capable of binding a functional biological molecule, the substrate comprising a hydrophilic surface activated to enable binding to said biological molecule and a sub-surface comprising a plurality of cross-linked regions.

According to another embodiment of the present invention there is provided a device comprising a polymer substrate functionalised with a functional biological molecule, the functionalised polymer substrate comprising a hydrophilic surface with a biological molecule bound thereto and a sub-surface comprising a plurality of cross-linked regions.

According to another embodiment of the present invention there is provided a method of producing an activated polymer substrate comprising exposing a surface of a polymer substrate to plasma treatment with a suitable plasma forming gas, under plasma immersion ion implantation conditions.

According to another embodiment of the present invention there is provided an activated polymer substrate produced according to a method comprising exposing a surface of a polymer substrate to plasma treatment with a suitable plasma forming gas, under plasma immersion ion implantation conditions.

According to another embodiment of the present invention there is provided a method of producing a polymer substrate functionalised with a biological molecule, comprising steps of:
(a) exposing a surface of a polymer substrate to plasma treatment with a suitable plasma forming gas, under plasma immersion ion implantation conditions;
(b) incubating the surface treated according to step (a) with a desired biological molecule.

According to another embodiment of the present invention there is provided a polymer substrate functionalised with a biological molecule produced according to a method comprising steps of:
(a) exposing a surface of a polymer substrate to plasma treatment with a suitable plasma forming gas, under plasma immersion ion implantation conditions;
(b) incubating the surface treated according to step (a) with a desired biological molecule.

### BRIEF DESCRIPTION OF THE FIGURES

The invention will be further described with reference to the figures, wherein:

FIG. 1 shows a schematic diagram of the inductively coupled plasma treatment chamber useful in methods of activating surfaces of polymer substrates according to the invention.

FIG. 2 is demonstrative of decay of surface hydrophilicity (recovery of hydrophobicity) over time following plasma treatment and shows a graph of water contact angle (degrees) against time (days) for untreated (filled square) and treated (un-filled square) polyethylene (PE) samples.

FIG. 3 is demonstrative of horseradish peroxidase (HRP) attachment and activity on PE samples. It shows a graph of optical density (O.D.) at 450 nm against time (days) for PE surfaces incubated with PO₄ (diamonds) and PBS (squares) buffers and both plasma treated under PIII conditions (un-filled) and untreated (filled). The same buffers were used for making up the HRP containing solution in which the samples were soaked over night before the first measurement and for washing, which was carried out on a daily basis thereafter.

FIG. 4 is demonstrative of horseradish peroxidase (HRP) attachment and activity on PE samples in the presence of blocking detergent (Tween 20). It shows a graph of optical density (O.D.) at 450 nm against time (days) for PE surfaces incubated with PBS-Tween buffer (used for making up the HRP containing solution), washed with either PO₄ (diamonds) or PBS-Tween (squares) buffers and data for both plasma treated under PIII conditions (un-filled) and untreated (filled) surfaces. Note that the two results sets for the untreated samples are overlapping.

FIG. 5 is demonstrative of levels of horseradish peroxidase (HRP) attachment and activity on PE samples using different HRP concentrations. It shows a graph of optical density (O.D.) at 450 nm against HRP incubation concentration (mg/ml) for both plasma treated under PIII conditions (un-filled) and untreated (filled) and both with dilution (triangles) and without dilution (squares) when measuring optical density.

FIG. 6 is demonstrative of catalase attachment and activity (by assaying hydrogen peroxide, which is decomposed in a reaction catalysed by catalase) and also demonstrates improved binding stability of surfaces treated under plasma immersion ion implantation conditions. It shows a graph of optical density (O.D.) at 475 nm against time (days) for PE surfaces either untreated (square containing cross), plasma treated (circle) or plasma treated under plasma immersion ion implantation conditions (filled square) and then incubated with catalase and washed daily thereafter. A control comprising the same buffer and 6 mM hydrogen peroxide is shown with filled circles.

FIG. 7 shows a bar graph of optical density using the HRP assay at day 0 (shaded) and day 3 (unshaded) for the plasma (using PIII conditions) treated and untreated competitor polymer surfaces, nunc and HTA, as well as for both untreated polyethylene (PE) and PE plasma treated under PIII conditions.

FIG. 8 shows plots of absorbance at 475 nm against time to demonstrate the effect of Tween 20 on the attachment of catalase to nitrogen treated polyethylene. A Surface was treated with Tween 20 for 1 hour before addition of the catalase/Tween solution. B Catalase/Tween added without prior incubation of the surface with Tween: (filled square) PIII; (square containing cross) Plasma; (filled circle) untreated; (empty circle) 6 mM H₂O₂.

FIG. 9 shows a plot of absorbance at 475 nm against NaCl concentration (moles/l) to demonstrate the effect of sodium chloride on the attachment of catalase to nitrogen treated polyethylene. Absorbance of 6 mM H₂O₂=1.20±0.08: (filled square). PIII; (square containing cross) Plasma; (filled circle) untreated; (empty circle) 6 mM H₂O₂.

FIG. 10 shows a plot of absorbance at 475 nm against the logarithm of catalase concentration (μg/ml) to demonstrate the effect of increasing concentrations of catalase on functional attachment to nitrogen treated polyethylene, Equations for line of best fit up to 50 μg/ml catalase show rate of attachment greater for plasma treated surfaces. PIII y=0.816 (±0.048) 0.23 (±0.044) x; plasma y=0.78 (±0.042)−0.27 (±0.039) x; untreated y=0.944 (±0.055)−0.16 (±0.051) x. Absorbance of 6 mM H₂O₂=1.00±0.044: (filled square) PIII; (square containing cross) Plasma; (filled circle) untreated; (empty circle) 6 mM H₂O₂.

FIG. 11 shows graphs of optical density measurements from the HRP activity assay on PE surfaces incubated within 5 hours of the treatment process (▪), 2 weeks after treatment (), and 4 weeks after treatment (▴). (a) shows results for the argon plasma treatment process and (b) shows results for the argon PIII surface treatment. The points are the mean of three measurements and the error bars correspond to 1 standard deviation. The day 0 data points correspond to activity measured on samples which were incubated in HRP solution overnight and then washed in fresh buffer before testing by an activity assay.

FIG. 12 shows graphs of optical density measurements as in FIG. 11 from HRP activity assays on PE surfaces incubated in HRP solution within 5 hours of the treatment process (▪), 4 weeks (▴), 6 months () and 1 year (Δ) after treatment. Since the assays were done at different times, one set of control samples was analysed together with each, hence three sets of data for untreated surfaces are shown in the figure. The controls are shown on the left; the PE surfaces treated with the nitrogen PIII process on the right and the nitrogen plasma treated surfaces in the centre.

FIG. 13 shows a bar graph of absorbance against time (days), demonstrating the time period of retention of functional protein (Soybean Peroxidase (SBP)) on the Polystyrene (PS) surface over time, where untreated (black), plasma treated (white) and PIII treated (grey) surfaces were stored in PB for the duration of the experiment.

FIG. 14 shows a bar graph of absorbance against time (days), demonstrating the time period of retention of functional protein (Soybean Peroxidase (SBP)) on the Polystyrene (PS) surface over time, where untreated (black), plasma treated (white), PIII treated (grey) and PIII/O₂ plasma treated (thatched) surfaces were stored in PBS for the duration of the experiment.

FIG. 15 shows a bar graph of absorbance either following exposure to Tween 20 (grey) or not (black), for each of untreated, plasma treated and PIII-treated polystyrene surfaces incubated with soybean peroxidise.

FIG. 16 shows ATR-FTIR spectra taken of several PS surfaces, where all spectra shown are after subtracting out the original spectrum of the surface taken before soaking in SBP. (A) is of the untreated surface after incubating in protein (black) and after boiling in SDS (grey); (B) is of the PIII treated surface after incubating in protein (black) and after boiling in SDS (grey); and (C) is of various spectra after boiling the PIII treated surface in different solutions—SDS (from B), 1M HCl, 1M HCl+5% SDS and 1M NaOH+5% SDS.

FIG. 17 shows a schematic of a possible mechanism for covalent binding of protein to a PIII plasma-treated surface.

FIG. 18 shows ATR-FTIR spectra of treated, tris blocked PS surfaces after incubating in protein (SBP) (black) and after boiling in SDS (grey).

FIG. 19 shows a bar graph of absorbance either following incubation with SNA blocked tropoelastin (white), unblocked tropoelastin (grey) or no tropoelastin (black), for each of untreated and PIII-treated polystyrene surfaces.

FIG. 20 shows a bar graph of the percentage of human dermal fibroblast spreading for polystyrene surfaces either untreated or PIII treated, following tropoelastin coating at 0, 5 or 20 μg/ml and either with (filled) or without BSA blocking (cross-hatched).

FIG. 21 shows FTIR ATR spectra of PTFE after PIII treatment. From bottom to top: untreated, 5×10¹⁴ ions/cm², 10¹⁵ ions/cm², 2×10¹⁵ ions/cm², 5×10¹⁵ ions/cm², 10¹⁵ ions/cm².

FIG. 22 shows normalized absorbance of FTIR ATR spectral lines of PTFE after PIII treatment: rhomb—1882 cm⁻¹, cubic—1785 cm⁻¹, circle—1715 cm⁻¹, triangle—985 cm⁻¹.

FIG. 23 shows FTIR ATR subtracted spectra of PTFE after PIII treatment and HRP soaking minus PTFE after PIII treatment alone. From bottom: untreated, 5×10¹⁴ ions/cm², 10¹⁵ ions/cm², 2×10¹⁵ ions/cm², 5×10¹⁵ ions/cm², 10¹⁵ ions/cm².

FIG. 24 shows normalized absorbance of attached HRP lines in FTIR ATR spectra of PTFE after PIII treatment: triangle—1540 cm⁻¹ (Amide II) line, rhomb—3315 cm⁻¹ (Amide A) line.

FIG. 25 shows absorbance at 475 nm in TMB assay test for active HRP on PTFE surfaces after PIII treatment.

FIG. 26 shows a graph of absorbance at 450 nm against time (days) after freeze drying for untreated (filled circles), plasma (open squares) or PIII plasma (filled squares) treated polyethylene surfaces incubated with HRP.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Documents referred to within this specification are included herein in their entirety by way of reference.

The reference to any prior art in this specification is not, and should not be taken as, an acknowledgment or any form of suggestion that that prior art forms part of the common general knowledge in Australia.

As mentioned above, in one broad embodiment this invention relates to an activated polymer substrate capable of binding a functional biological molecule, the substrate comprising a hydrophilic surface activated to enable binding to said biological molecule and a sub-surface comprising a plurality of cross-linked regions. The invention also encompasses devices comprising such activated polymer substrates.

By the term “activated” it is intended to mean that the hydrophilic surface (which also results from the process of the invention) of the polymer substrate has been processed in a manner such that it is able to accept a biological molecule for binding, upon exposure thereto. That is, the surface of the polymer has one or more higher energy state regions where there are chemical groups or electrons available for participation in binding to one or more groups on a biological molecule, or indeed to suitable linker groups, which in turn are bound or are able to bind to a biological molecule.

In another broad aspect of the invention there is provided a polymer substrate functionalised with a functional biological molecule, the substrate comprising a hydrophilic surface activated to enable binding to said biological molecule and a sub-surface comprising a plurality of cross-linked regions. The invention also encompasses devices comprising such functionalised polymer substrates.

Without wishing to be bound by theory, the present inventors believe that through the activation of the polymer surface according to the invention it is possible to form chemical bonds, most likely covalent bonds, to chemical groups of biological molecules or linkers that attach to biological molecules. Preferably the chemical groups of the biological molecules are accessible for binding interactions, such as by being located on the exterior of the molecule. The present inventors believe that activation of the polymer surface involves the generation of reactive oxygen species, such as charged oxygen atoms and reactive carbonyl and carboxylic acid moieties that appear following exposure of the PIII plasma treated surface to oxygen (e.g. from air), and which are then available as binding sites for reactive species on biological molecules, such as amine groups.

Within this application we refer to attachment of a biological molecule, or a linker for attachment to a biological molecule, as functionalisation of the polymer substrate and to the polymer substrate to which the biological molecule or linker is attached as being “functionalised”. Attachment by covalent bonds to an otherwise strongly hydrophilic surface allows strong time stable attachment of biological molecules, that are able to maintain a useful biological function. For example, the hydrophilic surface of the polymer will ensure that it is not energetically favourable for proteins to denature on the surface. Covalent attachment to a surface can be achieved via amino acid side chain groups covalently attached to linker molecules, for example. The strategy adopted is to prepare the polymer surface with sites that encourage what is believed to be covalent attachment. A high energy ion treatment is utilised with the aim of stabilising the polymer surfaces simultaneously with the creation of the binding sites. Using functionality assays, the inventors have demonstrated that associated with the adopted plasma surface treatment there is enhancement of functional protein attachment, compared to non-treated surfaces, as well as significantly increased resistance to repeated washing steps. That is, there is increased biological molecule binding relative to non-treated surfaces, the binding is strong and can withstand repeated washing and the molecule is able to retain useful activity (ie. the biological molecule is functional or retains some useful functionality).

By the term “functional” it is intended to convey that the molecule is able to exhibit at least some of the activity it would normally exhibit in a biological system. For example, activity may include the maintained ability to participate in binding interactions, such as antigen/antibody binding, receptor/drug binding or the maintained ability to catalyse or participate in a biological reaction, even if this is at a lower level than is usual in a biological system. Routine assays are available to assess functionality of the biological molecule. Preferably the activity of the biological molecule bound to the activated polymer surface is at least 20%, preferably at least 40%, more preferably at least 60%, 70% or 80% and most preferably at least 90%, 95%, 98% or 99% of the activity of the molecule when not bound to the activated polymer. Most preferably the activity of the bound biological molecule is equivalent to that of a non-bound molecule.

By the term “biological molecule” it is intended to encompass any molecule that is derived from a biological source, is a synthetically produced replicate of a molecule that exists in a biological system, is a molecule that mimics the activity of a molecule that exists in a biological system or otherwise exhibits biological activity. Examples of biological molecules include, but are not limited to, amino acids, peptides, proteins, glycoproteins, lipoproteins, nucleotides, oligonucleotides, nucleic acids (including DNA and RNA), lipids and carbohydrates, as well as active fragments thereof. Preferred biological molecules include proteins and drugs or drug targets. Particularly preferred biological molecules include antibodies and immunoglobulins, receptors, enzymes, neurotransmitters or other cell signalling agents, cytokines, hormones and complimentarily determining proteins, and active fragments thereof. The term “biological molecule” also encompasses molecules that are integral to or attached to cells or cellular components through which cells or cellular components may be bound to the activated polymer. Further specific examples of biological molecules included within the invention are toxins and poisons including naturally occurring toxins such as bacterial, plant or animal derived toxins or active fragments thereof including conotoxin and snake and spider venoms, for example, and other organic or inorganic toxins and poisons such as cyanide and anti-bacterial, anti-fungal, herbicide and pesticide agents.

An advantage associated with the present invention is that the process for binding biological molecules to the surface of a polymer does not depend upon the specific biological molecule or polymer and can therefore be applied to a wide variety of biological molecules and polymers. Furthermore, and although it is possible for the biological molecules to be bound via a linker molecule, it is not necessary according to the present invention for linker molecules to be utilised, which means that time consuming and potentially costly and complex wet chemistry approaches for linkage are not required.

As indicated above the present invention can be utilised to attach functional biological molecules to surfaces of a wide variety of polymer substrates. For example the polymer substrate may take the form of a block, sheet, film, strand, fibre, piece or particle (eg. a nano- or micro-particle such as a nano- or micro-sphere), powder, shaped article, woven fabric or massed fibre pressed into a sheet (for example like paper) of homo-polymer, co-polymer, polymer mixture or polymer containing material. The polymer substrate can be a solid polymeric mono-material, laminated product, hybrid material or alternatively a coating on any type of base material which can be non-metallic or metallic in nature. Indeed, the polymer substrate may also form a component of a device, such as for example a component of a diagnostic kit, a tissue or cell culture scaffold or support, a biosensor, an analytical plate, an assay component or a medical device such as a contact lens, a stent (eg a cardiovascular or gastrointestinal stent), a pace maker, a hearing aid, a prosthesis, an artificial joint, a bone or tissue replacement material (e.g. replacement skin, connective tissue, muscle or nerve tissue), an artificial organ or artificial skin, an adhesive, a tissue sealant, a suture, staple, nail, screw, bolt or other device for surgical use or other implantable or biocompatible device.

Other devices that may be produced according to the invention are those related to chemical processing. For example, the invention includes devices utilised in chemical processes conducted on surfaces or substrates that may result in generation of fuels, biofuels, electricity or production of chemical products (e.g. bulk or fine chemicals, drugs, proteins, peptides, nucleic acids, polymers, food supplements and the like). In a preferred embodiment the invention includes devices used in the production of ethanol by the action of enzymes on sugars or cellulose or other agents. The invention also includes devices used in production of electricity by means of a chemical reaction catalysed by an enzyme, such as in a fuel cell or bio-fuel cell. In this context the invention provides surfaces functionalised by enzymes that can be made available to chemical agents to be processed by immersion in them or by arranging for the agents to flow over the surfaces. In the case that the agent flows over the enzyme-functionalised surface, problems with the poisoning of the enzyme by the products of the reaction can be minimised. Another advantage of the invention is that the enzyme functionalised surface can be rapidly and conveniently replaced with another fresh functionalised surface in the event that the enzymes become poisoned or are otherwise rendered inactive, without the need to dispose of the entire batch of chemicals.

As a result of the plasma treatment according to the invention under plasma immersion ion implantation (PIII) conditions the present inventors have determined that not only is the polymer surface activated to allow binding of one or more biological molecules, but that the generally hydrophobic nature of the polymer surface is modified to exhibit a more hydrophilic character. This is important for maintaining the conformation and therefore functionality of many biological molecules, the outer regions of which are often hydrophilic in nature due to the generally aqueous environment of biological systems. The inventors have also shown that not only do techniques of the present invention give rise to hydrophilicity of the treated polymer surfaces, but that as a result of the PIII treatment conditions there is a delay to the hydrophobic recovery of the surface that takes place over time following the treatment, relative to polymer surfaces that are plasma treated but without PIII conditions. The inventors understand that the mechanism associated with delayed hydrophobic recovery is that in addition to the treatment giving rise to surface activation it also results in improved surface stabilisation. This stabilisation is understood to result from penetration into the sub-surface of the polymer of energetic ions, giving rise to regions of polymer cross-linking in the substrate sub-surface. Although the polymer surface is likely to be rough on an atomic scale, meaning that it is difficult to define the surface as a smooth plane, the energies of ions utilised will ensure that they penetrate at least about 1 nm into the interior of the polymer and up to about 300 mm. It is therefore intended for the term “sub-surface” to encompass a region of the polymer that is between about 1 mm and about 300 nm beneath the surface subject to plasma treatment under PIII conditions, preferably between about 5 nm and about 200 nm, and most preferably between about 10 nm and about 100 nm beneath the surface.

The term “polymer” as it is used herein, is intended to encompass homo-polymers, co-polymers, polymer containing materials, polymer mixtures or blends, such as with other polymers and/or natural and synthetic rubbers, as well as polymer matrix composites, on their own, or alternatively as an integral and surface located component of a multi-layer laminated sandwich comprising other materials e.g. polymers, metals or ceramics (including glass), or a coating (including a partial coating) on any type of substrate material. The term “polymer” encompasses thermoset and/or thermoplastic materials as well as polymers generated by plasma deposition processes.

The polymeric materials which can be treated according to the present invention include, but are not limited to, polyolefins such as low density polyethylene (LDPE), polypropylene (PP), high density polyethylene (HDPE), ultra high molecular weight polyethylene (UHMWPE), blends of polyolefins with other polymers or rubbers; polyethers, such as polyoxymethylene (Acetal); polyamides, such as poly(hexamethylene adipamide) (Nylon 66); polyimides; polycarbonates; halogenated polymers, such as polyvinylidenefluoride (PVDF), polytetra-fluoroethylene (PTFE) (Teflon™), fluorinated ethylene-propylene copolymer (FEP), and polyvinyl chloride (PVC); aromatic polymers, such as polystyrene (PS); ketone polymers such as polyetheretherketone (PEEK); methacrylate polymers, such as polymethylmethacrylate (PMMA); polyesters, such as polyethylene terephthalate (PET); and copolymers, such as ABS and ethylene propylene diene mixture (EPDM). Preferred polymers include polyethylene, PEEK and polystyrene.

The term “plasma” or “gas plasma” is used generally to describe the state of ionised gas. A plasma consists of charged ions (positive or negative), negatively charged electrons, and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and gas at low pressure is bled into the system. An electromagnetic field generated by a capacitive or inductive RF electrical discharge is used to ionise the gas. Free electrons in the gas absorb energy from the electromagnetic field and ionise gas molecules, in turn producing more electrons.

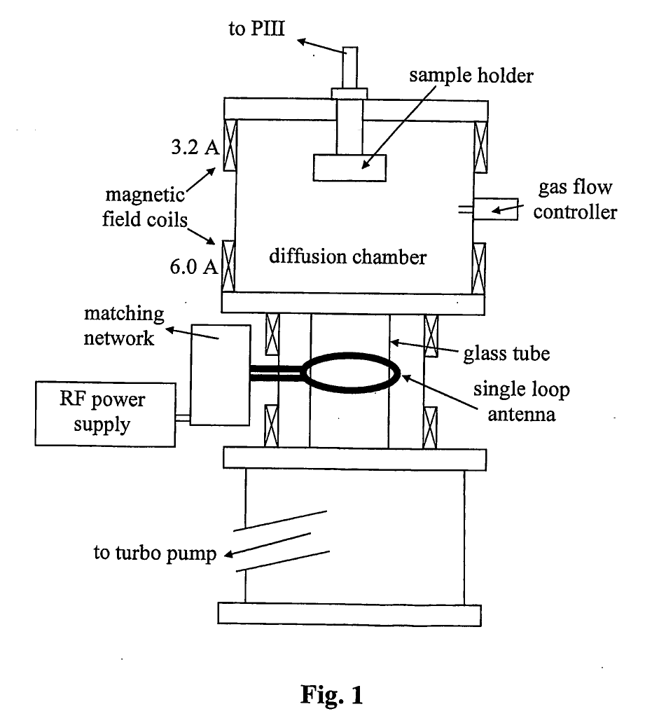

In conducting the plasma treatment according to the invention, typically a plasma treatment apparatus (such as one incorporating a Helicon plasma source or other inductively or capacitively coupled plasma source, such as shown in FIG. 1) is evacuated by attaching a vacuum nozzle to a vacuum pump. A suitable plasma forming gas from a gas source is bled into the evacuated apparatus through a gas inlet until the desired gas pressure in the chamber and differential across the chamber is obtained. An RF electromagnetic field is generated within the apparatus by applying current of the desired frequency to the electrodes from an RF generator. Ionisation of the gas in the apparatus is induced by the electromagnetic field, and the resulting plasma in the tube modifies the polymer substrate surface subjected to the treatment process.

Suitable plasma forming gases used to treat the surface of the polymer substrate include inorganic and/or organic gases. Inorganic gases are exemplified by helium, argon, nitrogen, neon, water vapour, nitrous oxide, nitrogen dioxide, oxygen, air, ammonia, carbon monoxide, carbon dioxide, hydrogen, chlorine, hydrogen chloride, bromine cyanide, sulfur dioxide, hydrogen sulfide, xenon, krypton, and the like. Organic gases are exemplified by methane, ethylene, benzene, formic acid, acetylene, pyridine, gases of organosilane, allylamine compounds and organopolysiloxane compounds, fluorocarbon and chlorofluorocarbon compounds and the like. In addition, the gas may be a vaporised organic material, such as an ethylenic monomer to be plasma polymerised or deposited on the surface. These gases may be used either singly or as a mixture of two more, according to need. Preferred plasma forming gases according to the present invention are nitrogen and argon.

Typical plasma treatment conditions (which are quoted here with reference to the power that may be required to treat a surface of 100 square centimetres, but which can be scaled according to the size of the system) may include power levels from about 1 watt to about 1000 watts, preferably between about 5 watts to about 500 watts, most preferably between about 10 watts to about 100 watts (an example of a suitable power is forward power of 100 watts and reverse power of 12 watts); frequency of about 1 kHz to 100 MHz, preferably about 15 kHz to about 50 MHz, more preferably from about 1 MHz to about 20 MHz (an example of a suitable frequency is about 13.5 MHz); axial magnetic field strength of between about 0 G (that is, it is not essential for an axial magnetic field to be applied) to about 100 G, preferably between about 20 G to about 80 G, most preferably between about 40 G to about 60 G (an example of a suitable axial magnetic field strength is about 50 G); exposure times of about 5 seconds to 12 hours, preferably about 1 minute to 2 hours, more preferably between about 5 minutes and about 20 minutes (an example of a suitable exposure time is about 13 minutes); gas pressures of about 0.0001 to about 10 torr, preferably between about 0.0005 torr to about 0.1 torr, most preferably between about 0.001 torr and about 0.01 torr (an example of a suitable pressure is about 0.002 torr); and a gas flow rate of about 1 to about 2000 cm³/min.

According to the present invention the plasma treatment will be under plasma immersion ion implantation (PIII) conditions, with the intention of implanting the sub-surface of the polymer substrate with the gas species. Typical PIII conditions include a substrate bias voltage to accelerate ions from the plasma into the treated polymer of between about 0.1 kV to about 150 kV, preferably between about 0.5 kV to about 100 kV, most preferably between about 1 kV to about 20 kV (an example of a suitable voltage is about 10 kV); frequency of between about 0.1 Hz to about 1 MHz, preferably between about 1 Hz to about 100 Hz, most preferably between about 20 Hz to about 80 Hz (an example of a suitable frequency is about 50 Hz); pulse-length of between about 1 μs to about 1 ms, preferably between about 10 μs to about 100 μs (an example of a suitable pulse-length is about 20 μs).

Following activation of the polymer substrate surface it is possible to functionalise the polymer surface with a biological molecule or linker by simple incubation (eg. by bathing, washing or spraying the surface) of the activated surface (substrate) with a solution comprising the biological molecule or linker. Preferably the solution is an aqueous solution (eg. saline), that preferably includes a buffer system compatible with maintaining the biological function of the molecule, such as for example a phosphate or Tris buffer. It may then be appropriate to conduct one or more washing steps also using a biologically compatible solution or liquid, for example the same aqueous buffered solution as for the incubation (but which does not include the biological molecule), to remove any non-specifically bound material from the surface, before the functionalised polymer substrate is ready to be put to its intended use.

The inventors have determined that both the activated polymer substrates and the polymer substrates functionalised with biological molecules according to the invention exhibit extensive shelf life. For example, the activated polymer substrate may be stored (preferably in a sealed environment) for a period of minutes, hours, days, weeks or months before incubation with a biological molecule to result in functionalisation of the polymer surface. Similarly the polymer substrates functionalised with biological molecules according to the invention may be stored (preferably following freeze drying and more preferably in a sealed environment at low temperature) for periods of minutes, hours, days, weeks, months or years without significant degradation before being re-hydrated, if necessary, and put to their intended use. If freeze drying is adopted a stabiliser such as sucrose may beneficially be added before the freeze drying process. The sealed environment is preferably in the presence of a desiccant and may comprise a container or vessel (preferably under vacuum or reduced oxygen atmosphere) or may for example comprise a polymer, foil and/or laminate package that is preferably vacuum packed. Preferably the sealed environment is sterile to thus prevent or at least minimise the presence of agents such as proteases and nucleases that may be detrimental to activity of the biological molecules.

The invention will now be described further, and by way of example only, with reference to the following non-limiting examples.

### EXAMPLES

### Example 1

### Plasma Treatment of Polyethylene for Enhanced Binding of Functional Horseradish Peroxidase

### Materials and Methods

FIG. 1 shows a schematic of the plasma treatment chamber. The source region consists of a single loop antenna, 16 cm diameter, wrapped around a boro-silicate glass tube. Radio frequency power at 13.56 MHz is coupled to the antenna by a Comdel CPM-2000 matching network. An aluminium diffusion chamber located above the plasma source houses the sample holder. The outside of the aluminium chamber is surrounded by 2 pairs of copper coils, used to provide an axial magnetic field of approximately 50 G.

The base pressure of the chamber is around 3×10⁻⁶ torr. Nitrogen gas was injected into the vacuum chamber to a pressure in the chamber of around 2 mT. The forward power used in the plasma chamber was 100 W, matched with a reverse power of 12 W. The technique of Plasma Immersion Ion Implantation (PIII) was used with conditions of 20 kV, 50 Hz and a pulse length of 20 μs. Polymer samples were treated using these conditions for a duration of 13 mins and 20 secs, giving an implanted ion fluence of approximately 10¹⁶ ions·cm⁻².

The polymer treated was Ultra High Molecular Weight Polyethylene (UHMW PE) film, with a thickness of approximately 200 μm. The polymer was sourced from Goodfellow Cambridge Limited, cat no ET301200/1. The polymer sheet was cut into 10 mm×13 mm rectangular samples. Contact angles were measured before and after plasma treatment using de-ionised water on a Kruss contact angle apparatus, (DS10). Measurements were taken as an average of 3 droplets.

After treatment samples were incubated with the protein horseradish peroxidase (HRP) within 2 hours of venting the vacuum chamber to air. The HRP was from Sigma, P6782. Three different buffers were used for both making up HRP containing solutions and for washing: (i) 10 mM phosphate (PO₄), pH 7 (ii) Phosphate Buffer Saline (PBS) buffer, pH 7.3 (concentration of salt 150 mM) and (iii) Phosphate Buffer Saline+Tween 20 detergent at 1 mg·ml⁻¹ (PBS-T). Tween 20 was from BDH Chemicals, Australia. Unless otherwise stated, the HRP concentration in the buffer solution was 50 ug·ml-1. The protein concentration was verified by absorption from the Heme group at 403 nm using the extinction coefficient of 102 mM·cm⁻¹ [1].

After overnight incubation in the HRP buffer solution, samples were washed 6 times for 20 minutes in fresh buffer solution. Untreated samples were used as controls. After washing, each sample was clamped between two stainless steel plates separated by an O ring (inner diameter 8 mm, outer diameter 11 mm) which sealed to the plasma treated sample surface. The top plate contained a 5 mm diameter hole, enabling the addition of 75 μl TMB (3,3′,5,5′ tetramethylbenzidine, Sigma T0440), an HRP substrate, to an area of polymer surface determined by the diameter of the O ring. After 30 secs, 50 μl aliquots were taken and added to 50 μl of 2 M HCl, in a 100 μl cuvette to stop the reaction. The optical density (O.D.) at a wavelength of 450 nm was measured in transmission through the cuvette using a DUO 530 Life Science UV/VIS spectrophotometer. Each data point presented was the average of measurements taken from at least 3 samples.

### Results and Discussion

In order to ensure that the optical density (O.D.) measured in our HRP functionality assay could not be due to chemical processes occurring on the buffer soaked polymer surfaces, the assay was carried out in PBS and PO₄ buffers in the absence of HRP. In both cases the optical densities measured were zero.

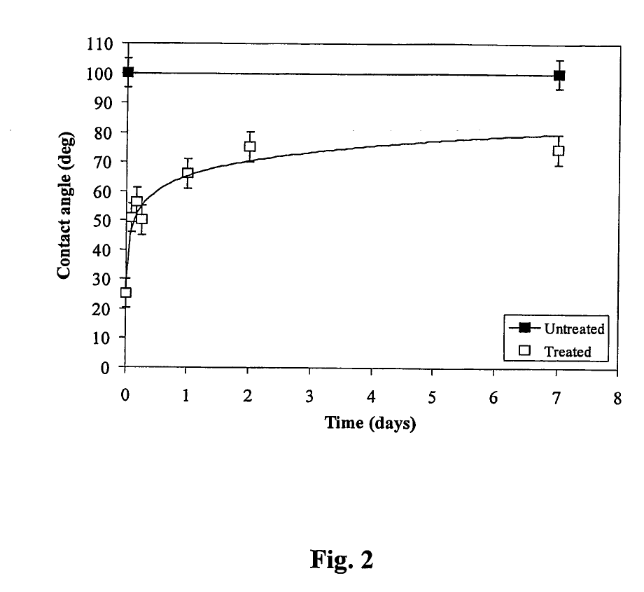

FIG. 2 shows the water contact angle as measured on plasma treated PE surfaces as a function of time after removal from the vacuum chamber in which the treatment was carried out and exposure to atmosphere. The plasma surface treatment makes the surface more hydrophilic than the untreated surface. Although the surface undergoes a significant hydrophobic recovery over time it did not relax back to its original contact angle during our observation time.

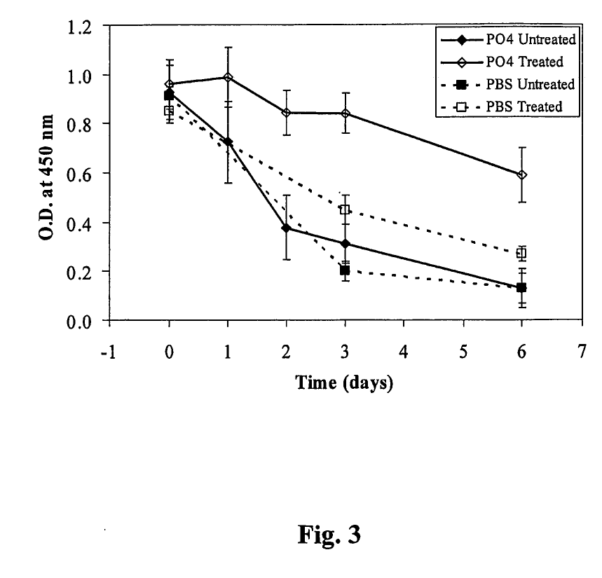

FIG. 3 shows optical density measurements from our HRP functionality assay as a function of time. HRP was bound by soaking the samples overnight in HRP containing buffer (either PBS or PO₄ as indicated). The first functionality assay (data point at day 0) was carried out immediately after six 20 minute washes in clean buffer. The first data point in each set is close to the saturation level of the O.D. measurement so it is difficult to compare activity between samples after the overnight soaking and first washing steps.

Subsequent data points represent another cycle of six 20 minute washes followed by application of the functionality assay. A clear result of these experiments is the significantly higher retention of activity after washing measured on the plasma PIII treated samples. Although increased retained protein activity is found on all of the plasma PIII treated samples compared to the untreated controls, the samples soaked and washed in PO₄ buffer significantly outperform those soaked and washed in the higher salt PBS buffer.

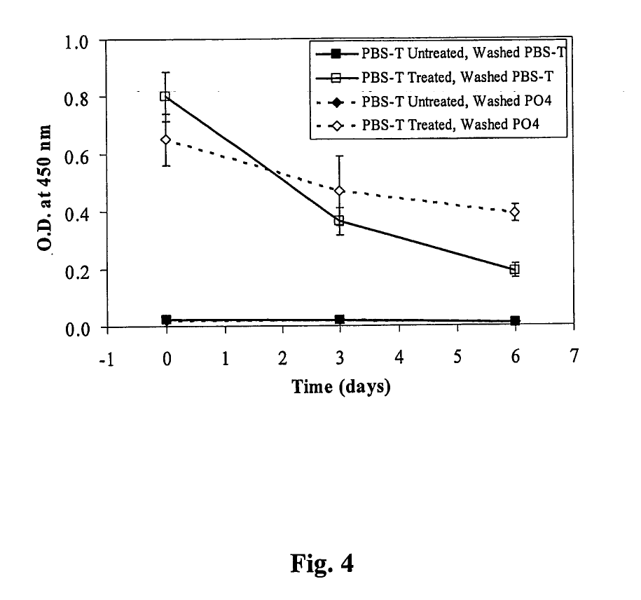

FIG. 4 shows the same experiments repeated with Tween 20 blocking detergent added to the buffer. The Tween 20 has a dramatic effect on the functional binding to the untreated surface but little impact on the results for the PIII treated surface, indicating that the mechanisms for binding on the two surfaces are very different and the affinity of the treated surface for HRP is substantially higher than that of the untreated surface. These results are also consistent with a protein binding mechanism on the treated surfaces which does not allow release of the protein after the initial surface attachment and allows retention of the protein conformation while bound to the surface.

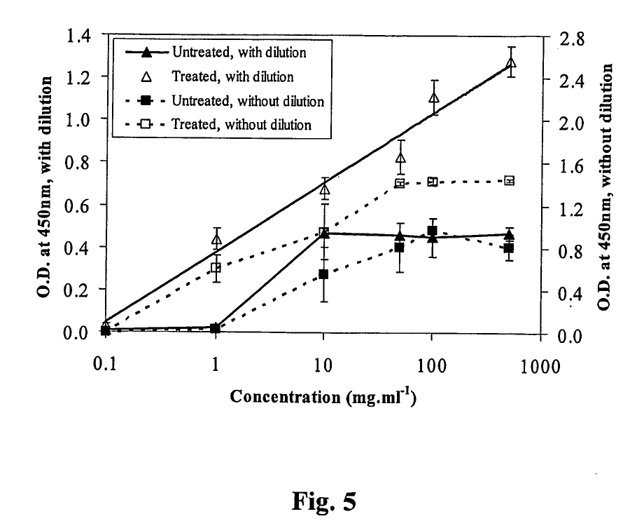

FIG. 5 shows the effect of HRP concentration in the buffer solution on the level of functional attachment after the first washing process. The set of measurements carried out with the higher level of dilution do not saturate while those carried out with the same dilution as used in FIGS. 3 and 4 saturate at an O.D. of around 1.4. This supports the assertion that the measurements of the first data points in FIG. 3 are likely to be saturated. The experiments at increased dilution show that the level of functional attachment increases in proportion to the logarithm of the concentration of the protein in solution. This result lends further support to the idea that, once attached to the active binding sites on the treated surface, the proteins remain attached to these sites. As the sites become occupied the density of sites available for attachment of subsequent HRP molecules decreases. The probability of binding is therefore reduced proportionally to the number of HRP molecules already bound to active sites.

The fact that significant functional binding is retained on the treated surfaces as compared to the untreated surfaces after repeated washing indicates that a new binding mechanism is introduced on the treated surfaces. This new binding is not blocked by Tween 20 as is the physisorption occurring on the untreated polymer. A model consistent with both of these results is that the plasma treatment creates active binding sites, which bind proteins in a manner by which their conformation and therefore their function are maintained over long time periods. Without wishing to be bound by theory, the plasma treatment may for example produce dangling bonds on the polymer surface which are able to covalently bind protein molecules. The covalent bonds do not interfere with the protein's function and are stable over time and resistant to washing.

Time stability of these bonds is also supported by the fact that the density of bound HRP increased linearly with the logarithm of the concentration in solution. This is consistent with a limited availability of covalent attachment sites, which become occupied by HRP molecules as soon as one of these molecules lands on that site. Once occupied, the sites do not release the HRP molecule and cannot accommodate further HRP. Thus the probability of a HRP molecule binding to the surface on impact will decrease with increasing coverage of the surface by HRP (hence a logarithmic relationship with concentration applies).

Although treated surfaces aged in air showed an increase in contact angle (or hydrophobic recovery), they did not show any changes in ability to bind functional HRP protein. This indicates that the binding mechanism associated with the plasma activated sites is not influenced by the hydrophobicity of the surface as would be expected from binding driven by a hydrophobic interaction. The result also indicates that the active sites introduced by the plasma treatment do not decay with time and are not affected by the changes associated with the change in contact angle over time.

In the absence of the Tween 20 blocker both the treated and untreated surfaces bind some proteins by non-specific physisorption. These protein molecules eventually lose their functionality or are washed away from the surface (the functionality assay can not distinguish between these two possibilities). They account for the gradual decay in active protein concentration seen on the treated and untreated surfaces in the absence of Tween 20.

The functional protein signal does not decay to zero over the measurement time of these experiments on the treated surfaces. This indicates that more stable binding sites relying on a significantly different binding mechanism are created by the plasma treatment. These sites have a high affinity for HRP molecules and retain the bound molecules in a functional state after repeated washing steps. A model of covalent attachment of the protein to dangling bonds or reactive centres, created by the energetic ion impacts of the plasma treatment or their subsequent oxidation in atmosphere, is consistent with these results.

### Example 2

### Plasma Treatment Under Plasma Immersion Ion Implantation Conditions of Polyethylene for Enhanced Binding of Functional Catalase

### Materials and Methods

The materials and methods adopted were the same as for Example 1, but with the exception that instead of HRP, plasma treated polymer surfaces were incubated with catalase (Sigma cat. no. C3155). An assay using surface exposure to hydrogen peroxide containing solution was then conducted according to the method of Cohen et al 2, as hydrogen peroxide is consumed in a reaction catalysed by catalase, to determine catalase functionality. The surface was incubated with 6 mM H₂O₂ and allowed to react for 6 minutes on an ELISA plate shaker, before an aliquot was taken and measured for remaining hydrogen peroxide. The remaining H₂O₂ was measured by adding excess ferrous ions, which are converted to ferric ions. Ferric ions were then reacted with thiocyanate to form a reddish/orange coloured complex which absorbs at a wavelength of 475 nm. The optical density at this wavelength thus provides a measure of the quantity of H₂O₂ remaining.

When optical density was measured the optical density of a 6 mM solution of hydrogen peroxide control solution was also measured.

### Results and Discussion

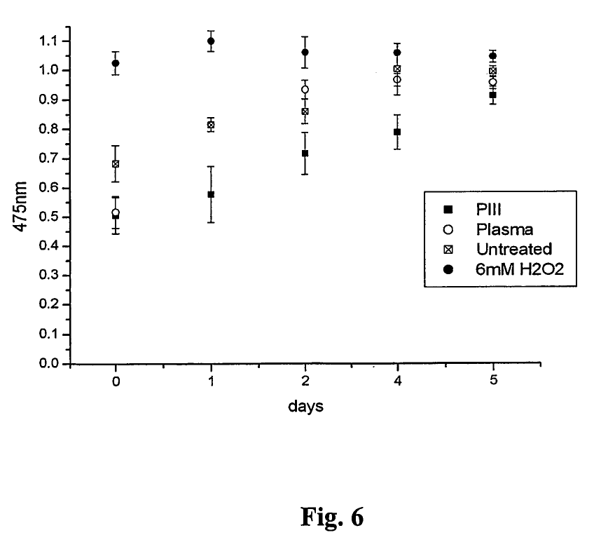

FIG. 6 shows that initial catalase functional binding to the treated polymer surfaces is greater than for non-treated surfaces. The functional binding is similar for surfaces treated with a simple RF discharge and for those treated also with PIII. FIG. 6 also demonstrates that activity of bound catalase is maintained at a higher level over the course of the experiment in the case of polymer surface treated with plasma under PIII conditions. Thus it is believed that plasma treatment under PIII conditions is more effective than simple plasma treatment in maintaining biological molecule functionality due to slowing of the rate of hydrophobic recovery of the treated polymer surface.

### Example 3

### Assessment of Enhanced Binding of Functional HRP to Polyethylene and Competitor Attachment Surfaces after Plasma Treatment Under Plasma Immersion Ion Implantation Conditions

### Materials and Methods

The materials and methods adopted were the same as for Example 1, but with the exception that the HRP functional binding assay was carried out on both PIII plasma treated and untreated competitor surfaces, nunc (Nunc MaxiSorb™ clear polymer microarray slides—ref 230302, from Nunc A/S, Denmark, www.nuncbrand.com) and HTA (HTA™ microarray slides from. Greiner Bio-One GmbH, Germany, www.greinerbioone.com) as well as for both treated and untreated polyethylene.

Optical density was measured after the first wash (as per day 0 points in example 1) and after three days with washing and buffer change each day. The error bars represent the standard deviation of the three measurements conducted for each experiment.

### Results and Discussion

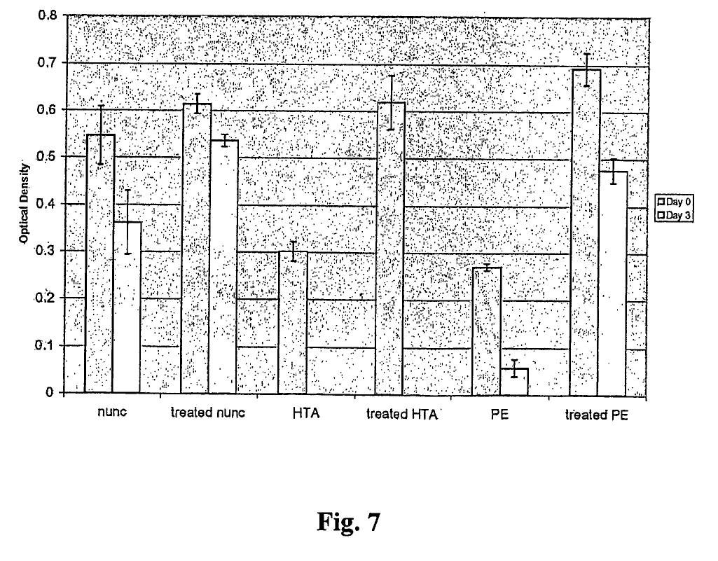

The results (as shown in FIG. 7) indicate that the plasma treatment process according to the invention produces increased binding against all control surfaces at day 0 and day 3. The improvement of nunc for day 0 is marginal and still within error bars for the sigma 1 confidence level, but the day three performance of the surface is significantly better. The conclusion for the treated nunc surface against untreated nunc is that the attachment performance is similar, but that the treatment enhances surface stability over time and repeated washing cycles. The treatment according to the invention when performed on the simple polyethylene surface gives rise to significantly improved functional HRP attachment compared to untreated nunc.

### Example 4

### Effect of Tween 20 on Functional Attachment of Catalase to Nitrogen Plasma Treated Polyethylene

### Materials and Methods

Catalase (Bovine liver catalase (EC 1.11.1.6) (C-3155, 20 mg/ml)) was attached to two sets of activated polyethylene surfaces using the same approach as for Example 2. One set of surfaces was treated with 10 mM PO₄ 0.005% Tween 20 (from BDH) for one hour whereas the other set was not treated with Tween 20. Catalase in 10 mM PO₄, 0.005% Tween 20 pH 7 was then added to both sets of surfaces and incubated overnight with rocking. Samples were then washed as in Example 1 with 10 mM PO₄ pH 7 buffer. No Tween 20 was included in the washing steps.

### Results and Discussion

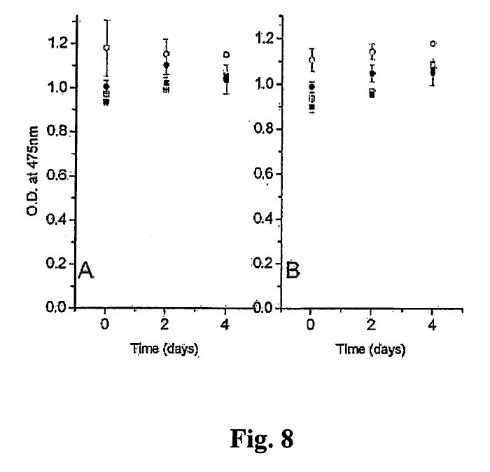

Detergents have long been used in ELISA assays for blocking areas of polymer surface not coated with bound antigen and for washing off loosely bound antigens, antibodies and reagents. In particular, non-ionic Tween 20 detergent has been widely used because it permanently blocks a surface and does not appear to affect the function of the protein under assay. The results of adding Tween 20 on the catalase functional assay are shown in FIG. 8. The blocking action was almost complete for untreated surfaces and both types of plasma treated surfaces. The same result of strong blocking occurred whether the surface was blocked first with Tween, or Tween and catalase were added simultaneously. To confirm that the effect was a blocking of attachment and not an inhibition of protein function, Tween 20 was added to catalase in solution and was found to have no adverse effect on the function of the enzyme. The experiment was also carried out in 10 mM PO₄ containing 0.15M NaCl at pH 7 and also in PBS buffer at pH 7.4 with and without added Tween 20. In both cases Tween 20 inhibited functional attachment to all surfaces (data not shown).

### Example 5

### Effect of Sodium Chloride on Functional Attachment of Catalase to Nitrogen Plasma Treated Polyethylene

### Materials and Methods

Catalase (Bovine liver catalase (EC 1.11.1.6) (C-3155, 20 mg/ml)) was attached to activated polyethylene surfaces using the same approach as for Example 2. Catalase was incubated in solutions of different NaCl concentrations overnight and washed as in Example 1, but in a solution of the same NaCl concentration that the protein was soaked in and where for the sixth wash the samples were transferred to new falcon tubes and all samples were washed in 10 mM PO₄.

### Results and Discussion

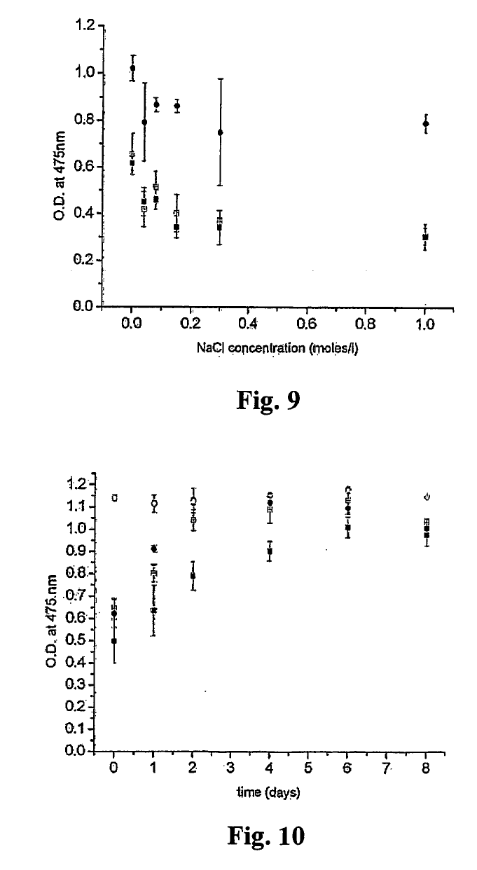

Electrostatic interactions between proteins and between proteins and surfaces are screened by the presence of ions in solution. To determine the role of electrostatic forces on the surface-protein interaction, we studied the effect of NaCl concentration on the attachment of catalase. The results in FIG. 9 show that increasing salt concentration did not reduce, but rather, increased the amount of functional activity on all of the surfaces. This implies that either more protein became attached or that the attached protein was better dispersed on the surface so its functional sites were more accessible. Catalase is known to aggregate in solution and perhaps higher salt concentrations could dissociate aggregates, resulting in a higher enzyme activity with the same amount of protein. The fact that the binding is not reduced in the presence of salt indicates that the interactions responsible for a large fraction of the binding are not of an electrostatic nature (ie. not based on charges and/or interactions between permanent dipoles). These results are consistent with covalent binding of catalase to the activated polyethylene surface.

### Example 6

### Effect of Surface Ageing on Functional Attachment of Catalase to Nitrogen Plasma Treated Polyethylene

### Materials and Methods

Catalase (Bovine liver catalase (EC 1.11.1.6) (C-3155, 20 mg/ml)) was attached to activated polyethylene surfaces using the same approach as for Example 2. Before conducting the catalase functional assay as in Example 2 the activated polyethylene samples were stored at room temperature for 4 months in a plastic container that was not airtight.

### Results and Discussion

FIG. 10 shows the functional attachment of catalase to plasma treated polyethylene after the treated samples were stored at room temperature for 4 months in a plastic container that was not airtight. The results for the stored treated surfaces were identical to samples that had catalase attached immediately after treatment. These results show that the plasma treatment is stable for at least 4 months. PIII treated samples showed superior attachment of functional protein at all time points, whereas plasma and untreated polyethylene were similar for the aged samples.

### Example 7

### Stability Over Time of Functional Horseradish Peroxidase Attached to Argon and Nitrogen Plasma Treated Polyethylene

### Materials and Methods

Polyethylene surfaces were exposed to plasma or PIII plasma treatment using high purity argon (22 standard cubic centimetres per minute (sccm)) or nitrogen (72 sccm), to incubation with HRP and activity assay under same conditions as for Example 1.

To assess the short term stability of the attached protein over time, samples exposed to argon plasma and argon PIII plasma treatment were kept in buffer solution which was replaced with fresh buffer each day. The assay was carried out on samples removed from the solution on the day following incubation (day 0), the day after that (day 1) and then every other day (days 3 and 5).

To assess the longer term stability (shelf life) of nitrogen plasma and PIII treated surfaces, the above procedure was repeated with surfaces that had been stored in a desiccator in dry air at room temperature and atmospheric pressure for 2, 4 weeks, 6 months and 1 year periods prior to incubation in the protein solution.

### Results and Discussion

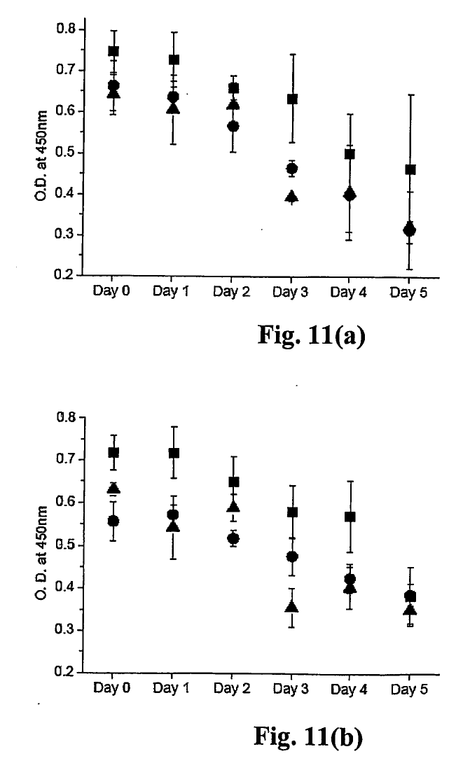

FIG. 11 shows the results from the HRP activity assay for samples stored in a desiccator for two and four weeks, compared with results from freshly treated samples. Both the fresh Ar plasma and Ar PIII treated surfaces show slightly higher levels of functional attachment than the aged samples in terms of the mean values plotted, but almost all of them agree within error bars (one standard deviation). The results demonstrate that any aging effect in treated samples is very small and has stabilised after 2 weeks.

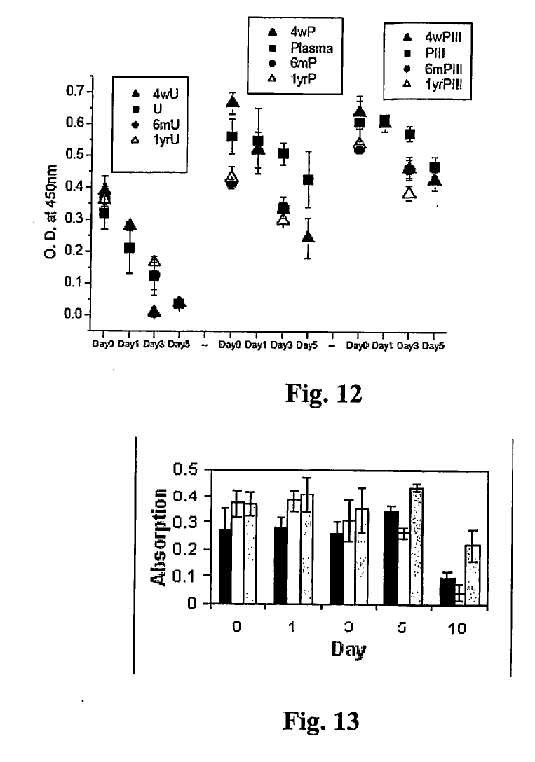

FIG. 12 shows the results from the HRP activity assay for samples stored in a desiccator for four weeks, six months and 1 year compared with results from freshly treated samples. The activity of the HRP attached to the plasma treated surfaces which had been stored for four weeks, six months and 1 year prior to incubation in protein solution decayed faster than that of HRP attached to the freshly treated samples. However, no significant difference in HRP activity was observed between the fresh PIII treated and the stored PIII treated surfaces.

These results demonstrate that the PIII-treated surfaces retained their properties for 2 to 4 weeks with only minimal loss of the protein binding and activity. The best performing treatment (plasma immersion ion implantation (PIII) using nitrogen plasma) showed no reduction in performance after 4 weeks and continued to show excellent binding and activity retention after one year of storage. In separate experiments this surface also exhibited the lowest water contact angle and the lowest level of hydrophobic recovery.

### Example 8

### Examination of Mechanism of Binding of Soybean Peroxidase to Plasma Treated Polystyrene Surfaces

### Materials and Methods

Polystyrene (PS) sheets (Goodfellow, 0.25 mm thick, biaxially oriented) were cut into small samples approximately 1 cm×1 cm in size. These samples were then cleaned with methanol and transferred into the plasma treatment chamber for treatment under the conditions outlined in Example 1. Two types of plasma treatment were applied. The first did not include the use of PIII to implant ions and the second applied PIII during the plasma treatment process. All protein attachment experiments were carried out on untreated control samples for comparison. In all cases involving a form of plasma treatment, the treatment time was 800 sec.

Phosphate buffer (PB) was 10 mM NaH₂PO₄ and 10 mM Na₂HPO₄, pH 7.0. Standard phosphate-buffered saline (PBS) was PB containing 150 mM NaCl adjusted to pH 7.4. Seed coat Soybean Peroxidase (SBP) was from Sigma-Aldrich and was chosen because its activity on a surface is easily determined by the use of a calorimetric assay. In the assay the reaction of a SBP substrate, 3,3′,5,5′-tetramethylbenzidine (TMB) is stopped with acid, forming a yellow reaction product, the optical density of which is read at 450 nm. Unlike horseradish peroxidase (HRP), SBP exists in only one isoform, and generally has greater stability.

Lyophilized SBP was reconstituted into buffer. The extinction coefficient ˜403=94.6 mM-1 cm-1 was then used to calculate the protein concentration⁴. The protein was then diluted with buffer to the concentrations used in the experiments.

After treatment, the samples and the untreated controls were incubated overnight in a solution of buffer containing SBP added to a concentration of 50 μg mL⁻¹ unless otherwise stated. The samples were then transferred to a new container and washed six times in fresh buffer solution, resting on a rocker for a period of 20 min for each wash. The samples were then stored in a tube in fresh buffer until they were measured using the TMB assay. If the samples were to be stored for longer periods, the solution was replaced with fresh buffer daily. The samples selected to be assayed on a given day were placed in small holders which consisted of two metal layers with a 7 mm diameter hole in the centre of one layer surrounded by a O-ring to seal the liquid in. 75 μL TMB was allowed to react for 30 sec, after which 50 μL were removed and acidified for spectrophotometry at 450 nm. The absorbance measured is related to the amount of functional protein on the surface. To determine relative estimates of the amount of protein (functional or not) left on the surface, infrared spectra were obtained using a Digilab FTS7000 FTIR spectrometer. The spectra were taken in attenuated total reflectance (ATR) mode using a multiple bounce germanium crystal, at a resolution of 1 cm⁻¹.

### Results and Discussion

FIG. 13 shows the results of a TMB activity assay on samples washed and stored over a 10 day period. Both the incubation and washing steps were done in phosphate buffer and the buffer was replaced daily. The initial (day 0) attachment of active protein appears to be enhanced somewhat on the two plasma treated surfaces. However, the surface treated using the PIII process shows much greater retention of active protein over the 10 day period compared to the untreated control and the surface treated without the PIII ion implantation process.

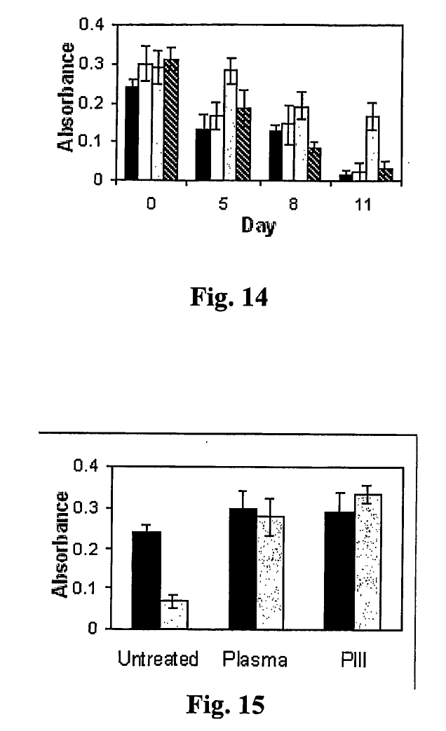

To test for any influence of the buffer choice we repeated the experiment with incubation in PBS solution containing the protein. The last washing step was done in PB, as salt is known to affect the TMB assay. The samples which were not to be assayed on a given day were stored in PBS until they were to be assayed. In addition to the two previously described treatment methods, another batch of samples was treated with the argon PIII process followed by a 10 second exposure to oxygen plasma. The results are shown in FIG. 14. The functional attachment at day 0 was enhanced for all three plasma treatments compared to the untreated control. Once again, little functional protein was retained on the untreated and plasma surfaces over time while the surface treated using the PIII process retained about half the amount of functional protein up to day 11. The subsequent exposure to oxygen plasma appears to have reduced the effectiveness of the PIII plasma treatment in terms of functional attachment over time.

The results shown in FIGS. 13 and 14 indicate that the surfaces exposed to the plasma as well as those exposed to the plasma with PIII treatment show an enhancement in initial binding. Only the PIII surface, however, has the ability to retain functional protein over longer periods. In the PIII treatment, ions penetrate many layers into the polystyrene, breaking and cross-linking the polymer chains at and below the surface. We believe that the formation of a cross-linked or carbonised layer just below the surface, preventing reptation of the modified polymer chains into the bulk, is the reason for the improved stability observed for the PIII treated surfaces.

In order to differentiate between binding mechanisms on the treated surfaces versus the untreated controls we washed the surfaces with detergent. The detergent Tween 20 is often used to block non-specific interactions. Surfaces were soaked in protein containing solutions overnight, and then washed with a solution of buffer containing 0.05% Tween 20. The results are shown in FIG. 15. The detergent removed most of the protein from the untreated surface, but not from either of the plasma treated surfaces, indicating that a stronger interaction which resists the detergent is responsible for at least some attachment on the plasma treated surfaces.

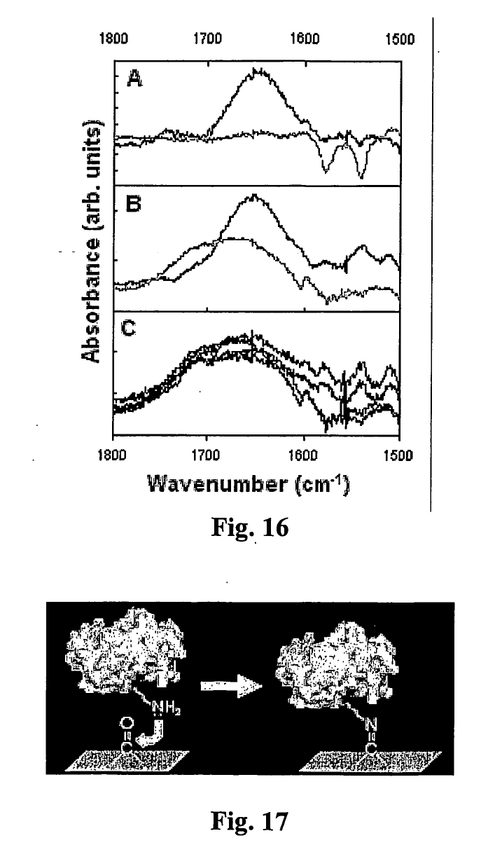

To further test the strength of attachment, the surfaces were boiled in a solution of 5% sodium dodecyl sulfate (SDS) for 10 minutes. The TMB assay was no longer effective, as SDS denatures the protein, so FTIR spectra of the surfaces were used to assess the quantity of protein remaining on the surfaces. While FTIR spectra of surfaces are often used to detect protein, the complexity of the spectrum of the underlying polystyrene made it difficult to see the peaks due to protein. To solve this issue, spectra of the surfaces were recorded both before and after incubation in protein, and then subtracted to give a difference spectrum. The resulting spectra as shown in FIG. 16 contain only peaks where the absorbance had changed from the original treated surface due to the attachment of protein. For both the treated and the untreated surfaces, amide peaks between 1650-1660 cm⁻¹ were present after incubation in protein solution, indicating the presence of protein.

Nothing in the spectrum of the buffer used had an absorption in this band. After boiling in 5% SDS, there was no amide peak present for the untreated surface, while it was still present on the surface treated using PIII, although reduced in intensity compared to the initial peak. The peak had also shifted slightly higher in wavenumber to approximately 1675 cm⁻¹, indicating unfolding of the protein as would be expected after boiling in SDS. SDS is therefore unable to detach all of the protein from the PIII treated surface as indicated by the continued presence of a peak associated with the protein. This is consistent with protein attachment through a covalent bond. To confirm this result, the surface was boiled in a solution containing both 5% SDS and 1 M NaOH. The protein still remained bound on the surface.

Using infrared spectral analysis of PIII treated polystyrene surfaces we have previously shown that upon atmospheric exposure, oxygen is absorbed and reacts with free radicals created by the treatment, generating carbonyl and other oxygen containing groups on the surface. It is known that carbonyls, for example, can form covalent bonds with amine groups through reactions such as Schiff base formation. A reaction of this type could be responsible for the observed covalent attachment of SBP to the PIII treated surfaces, as illustrated in FIG. 17. SBP has three exposed amino groups; two are in the form of lysines and one at the N-terminus. All three sites are on the opposite side of the protein from the active site, so attachment through these sites would not result in blocking the activity of SBP. Another possibility is direct attachment of the protein to the long-lived free radicals created in the plasma treatment process.

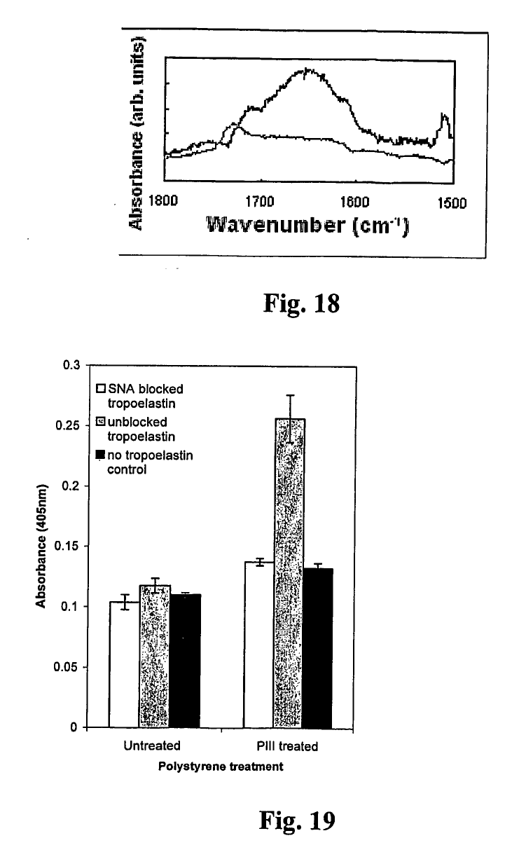

To test the idea that amine groups on the protein are involved in the new binding mechanism associated with the treated surfaces, samples were soaked for 3 days in 0.2 M tris(hydroxymethyl) aminomethane prior to exposure to SBP. The amine group of the Tris molecule would be expected to react with the active groups on the treated polymer surface, blocking these sites from subsequent interaction with the protein's amide groups. FTIR spectra were collected from the treated surface both before and after incubation in protein and then again after boiling in SDS. The spectra taken before incubation with protein were subtracted from those taken after incubation and after SDS exposure. The difference spectra obtained in this way are shown in FIG. 18. The spectra show that most, if not all protein was removed from the treated surface when boiled in SDS. This decrease in the amide peak is certainly much greater than the decrease from boiling the unblocked surface in SDS after incubation in protein solution. This result confirms that the covalent binding sites which bind the protein readily react with the C—NH₂ or the C—OH functional groups found in Tris, indicating that they would be likely to react with these groups in the protein. However, this still leaves open the possibility of the surface binding other functional groups on the protein as well.

Plasma treatment in an argon gas on polystyrene with concurrent PIII produces a surface with enhanced binding capacity for functional soybean peroxidase, as well as an enhanced ability to retain the protein function over time. The enhanced binding capacity seems to be at least in part due to the creation of functional groups which bind covalently to the protein. Advantages of using PIII to create functional sites for protein arrays and biosensors include the environmental friendliness and simplicity of the process, as well as its straight forward integration with currently existing methodologies for masking to create surface patterning. The process is completely dry, using only argon to create the functional sites, and no chemical linkers are needed to bind protein or other biological molecules.

An important aspect of creating protein arrays using a masked variant of this method for patterning will be minimising background protein adhesion to the untreated surface. These results show that a Tween 20 wash after incubation with protein gives a ratio of 1:5 in the functional protein remaining on the untreated and treated surfaces respectively. One strategy to eliminate the background signal is to treat the whole surface and mechanically place the protein on the desired sites, either through robotic placement, ink jet style protein printing³, or dip-pen nanolithography. The whole surface would then be blocked with an agent such as Tween 20, which is ineffective in removing protein that is bound to these treated surfaces.

### Example 9

### Examination of the Mechanism of Binding of Tropoelastin to PIII-Plasma Treated Polystyrene Surfaces by SNA Blocking of Amine Groups

### Materials and Methods

Polystyrene sheets (Goodfellows) were cut into 0.8×8 cm strips and wiped with 100% ethanol. Samples were mounted onto the target plate of a helicon PIII plasma chamber and PIII treated as described in Example 1. Untreated controls did not undergo treatment in the plasma chamber. Tropoelastin (produced in E. coli in-house by Professor Anthony Weiss⁵) was Sulfo-NHS Acetate (SNA) blocked as previously described (2). Briefly, tropoelastin was solubilised in 100 mM NaHCO₃, pH8.5 to 1 mg/ml and a 25-fold molar excess of SNA (Pierce) was added and incubated at room temperature for 1 hour. After incubation the excess SNA was removed by dialysis against 4×11 volumes of PBS at 4° C. A no SNA control was included alongside the SNA treated sample. Following dialysis the absorbance at 280 nm was measured and used to determine the tropoelastin concentration.

Strips of untreated, and PIII treated polystyrene were cut into 0.8×0.8 cm squares and placed into the wells of a 24 well plate (Greiner bio-one). SNA treated and untreated tropoelastin was diluted to 10 μg/ml in PBS and 0.75 ml added/well and incubated at 4° C. for 16 hours. Unbound tropoelastin was removed by aspiration, followed by 3×1 ml washes of PBS. The samples were SDS treated by transferring to 1.5 ml of 5% SDS (w/v) in PBS and incubated at 90° C. for 10 min. Following SDS treatment, the samples were placed into a 24 well plate, washed with 3×1 ml of PBS, and non-specific polystyrene binding was blocked with 10 mg/ml bovine serum albumin (BSA) (Sigma) in PBS for 1 hour at room temperature. Following BSA blocking the samples were washed with 2×1 ml PBS washes, then incubated in 0.75 ml of 1:1000 diluted mouse anti-elastin antibody (BA-4, Sigma) for 1 hour at room temperature. The antibody was removed, and the samples washed in 3×1 ml washes of PBS before incubation in 0.75 ml of 1:10000 diluted goat anti-mouse IgG-HRP conjugated secondary antibody (Sigma) for 1 hour at room temperature. The secondary antibody was removed and the samples washed with 4×1 ml PBS washes. The samples were transferred to a new 24 well plate and 0.75 ml of ABTS solution (Sigma) was added.

After 30-40 min the plates were agitated and 100 μl aliquots of the ABTS were transferred to a 96 well plate and the absorbance was read at 405 nm (BIORAD model 450 plate reader).

### Results and Discussion

As shown in FIG. 19 (where error bars indicate SD (n=3)) treatment with SDS completely removes non-SNA-blocked tropoelastin from untreated polystyrene to background levels. FIG. 19 also shows that SDS does not remove tropoelastin from PIII treated samples indicating that strong bonds exist between tropoelastin and PIII treated polystyrene. In contrast, SNA blocked tropoelastin was completely removed from both untreated and PIII treated polystyrene by SDS treatment, indicating that SNA has blocked side chains of tropoelastin involved in the strong interaction of tropoelastin with PIII treated polystyrene. As SNA blocks the amine groups of lysine side chains, this suggests that amines on lysine side chains are necessary for SDS resistant binding of tropoelastin to PIII treated polystyrene.

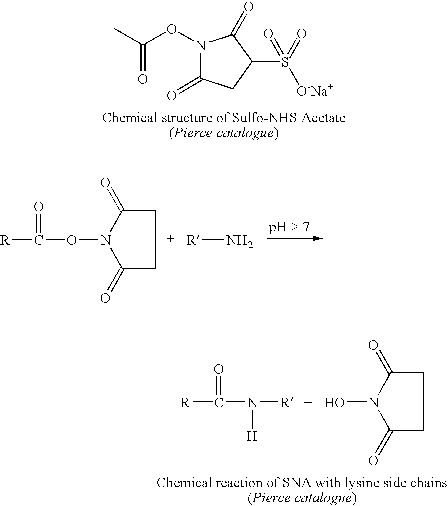

### Example 10

### Examination of Human Dermal Fibroblasts Spreading on Plasma Treated Polystyrene Surfaces Coated with Tropoelastin

### Materials and Methods

To determine cell spreading, 0.8×0.8 cm squares of untreated, and PIII treated (according to the procedure of Example 1) polystyrene were incubated in 0.75 ml of 10 μg/ml tropoelastin diluted in PBS in a 24 well plate at 4° C. for 16 hours. Unbound tropoelastin was aspirated, and cell binding to uncoated polystyrene was blocked with 10 mg/ml bovine serum albumin (BSA) (Sigma) in PBS for 1 hour at room temperature. Non-blocked samples were incubated in PBS without BSA. Near confluent 75 cm² flasks of human skin fibroblasts were trypsinized, by incubating with trypsin-EDTA (Gibco) at 37° C. for 4 minutes, followed by neutralization with equal volume of 10% FCS (Gibco) containing media (containing basal media (ICN biomedicals), non-essential amino acids (Gibco), essential amino acids (Gibco), and penicillin and streptomycin (Gibco)). The cell suspensions were centrifuged at 800 g for 3 minutes, and the cell pellets were resuspended in 5 ml of warm serum free media. The cell density was counted and adjusted to 1×10⁵ cells/ml. The BSA blocking solution was aspirated from the wells, followed by 3×1 ml washes of PBS. 0.75 ml aliquots of cells were added to the wells, then incubated at 37° C. in a 5% CO₂ incubator for 90 minutes. The cells were immediately fixed with the addition of 81 μl of 37% (w/v) formaldehyde (Sigma) directly to the well for 20 minutes. The formaldehyde was aspirated, and the wells filled with PBS before layering a glass plate onto the 24 well plate. The level of cell spreading was determined by phase contrast microscopy. Cells were spread when ‘phase-dark’ with visible nuclei, but un-spread when rounded and ‘phase-bright’.

### Results and Discussion

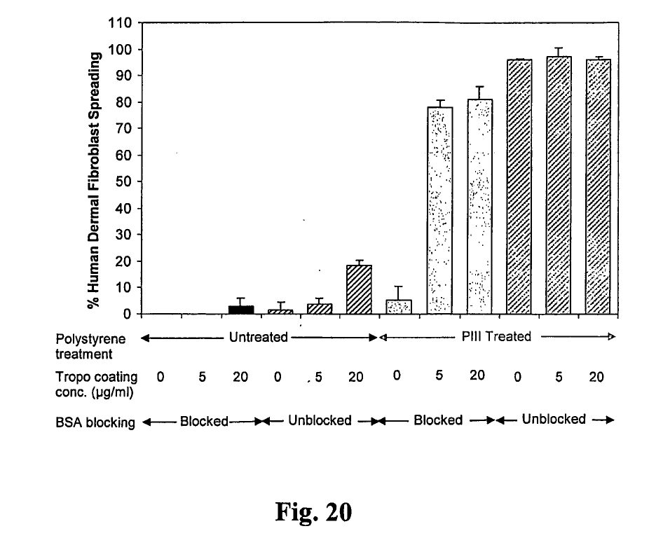

As can be seen from FIG. 20 (where error bars indicate SD (n=3)), human fibroblasts did not spread onto BSA blocked, untreated polystyrene in the absence of tropoelastin. FIG. 20 also shows that the tropoelastin coating only marginally increases cell spreading to 4% in the presence of BSA block, and to 18% in the absence of BSA block onto untreated polystyrene. BSA blocked PIII treated polystyrene supports 8% cell spreading, however in contrast to untreated polystyrene, tropoelastin coating dramatically increases cell spreading up to 80% and 83% for a tropoelastin coating concentration of 5 and 20 μg/ml, respectively. Cell spreading onto non-BSA blocked PIII treated polystyrene is very high at 98%, and so tropoelastin coating has no effect on the level of spreading.

PIII treatment significantly enhances cell binding on to the surface of polystyrene. In addition, PIII treatment also dramatically increases the level of cell spreading on to tropoelastin that is coated onto the polymer. This dramatic increase in cell binding could be due to a preferential morphology of the tropoelastin coated onto the surface, and/or due to changes in the hydrophobicity of the polystyrene, which allows for improved cell interactions. Therefore PIII treatment displays a dramatic improvement as it supports high cell binding to tropoelastin, which is strongly bound to the polymer surface.

### Example 11

### Assessment of Enhanced Binding of Functional HRP to PTFE (Teflon™) after Plasma Treatment Under Plasma Immersion Ion Implantation Conditions

### Materials and Methods

Horse Radish Peroxidase was purchased from Sigma (CAS Number: 9003-99-0, P6782) and dissolved in 10 mM phosphate (PO₄) buffer (pH 7) to a concentration of 1 μg·ml⁻¹. Polytetrafluorethylene (PTFE) of 20 μm thickness was from Halogen (Perm, Russia). Nitrogen gas used for the plasma treatment was 99.99% pure.

Plasma immersion ion implantation was carried out as in Example 1. The plasma density during treatment was continuously monitored using a Langmuir probe equipped with controller from Hiden Analytical Ltd. The samples were mounted on a stainless steel holder, with a stainless steel mesh, electrically connected to the holder, placed 45 mm in front of the sample surface. The samples were treated for durations of 20-800 secs, corresponding to implanted ion fluences of 0.5-20×10¹⁵ ions/cm². The ion fluence was calculated from the number of high voltage pulses multiplied by the fluence corresponding to one pulse. The fluence of one high voltage pulse was determined by comparing UV transmission spectra from satellite polyethylene films implanted under conditions used here to samples implanted with known fluences in previous PIII and ion beam treatment experiments.

The wettability of PTFE was measured using the sessile drop method, using Kruss contact angle equipment DS10 to measure the contact angles. For measurement de-ionised water, Glycerol, Formamide and Diiodomethane were dropped on the sample and the angle between edge of drop and the surface was measured. Surface energy and its components (polar and dispersic parts) were calculated using the Rabel model with regression method. After the PIII treatment the PTFE samples were stored for 3 days in air at room temperature (23° C.) in sealed containers. After 3 days the PTFE samples were incubated in HRP solution for 1 hour at 23° C. The solution contained HRP diluted to 50 ug/ml in 10 mM sodium phosphate buffer pH 7. After incubation, PTFE samples were washed six times in buffer (10 mM sodium phosphate buffer pH 7) for 2 hours each. Then samples for FTIR spectra were washed in de-ionised water for 10 seconds to remove buffer salts from the PTFE surface.

The PTFE samples (13 mm×15 mm) used in the TMB assay (not washed in de-ionised water) were clamped between two stainless steel plates separated by an O-ring (inner diameter 8 mm, outer diameter 11 mm) which sealed to the plasma treated surface. The top plate contained a 5 mm diameter hole. Hydrogen peroxide (75 μl, 6 mM) was added to the polymer surface and incubated for 6 minutes. During this time the plates were added to the surface of a tissue culture plate that was clamped to an ELISA plate shaker and shaken. After 6 minutes, 3 μl was removed and the remaining peroxide was assayed by a modified method of Cohen². The hydrogen peroxide was added to 0.25 ml of solution consisting of a mixture of 0.6M H₂SO₄ and 10 mM FeSO₄ and 20 μl of 2.5M KSCN was added to develop colour. Absorbance was measured at 475 nm using a DU 530 Beckman spectrophotometer.

FTIR ATR spectra of the PTFE samples were recorded using a Digilab FTS7000 FTIR spectrometer fitted with an ATR accessory (Harrick, USA) with a trapezium Germanium crystal at an incidence angle of 45°. To obtain sufficient signal/noise ratio and resolution of spectral bands 500 scans at a resolution of 1 cm⁻¹ were used. Before recording spectra, the surface of the PTFE was dried using dry air flow. Differential spectra of samples before and after PIII treatment as well as differential spectra of PIII treated samples with and without HRP attachment were used to detect changes. All spectral analysis was carried out using GRAMS software.

### Results and Discussion

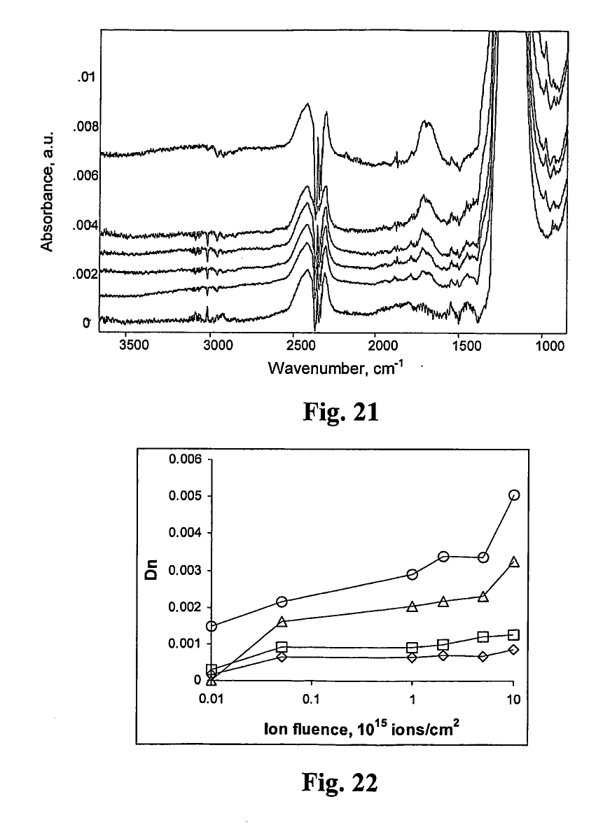

FIG. 21 shows spectra taken from the PTFE surface after PIII treatment. In comparison with untreated surface spectra, the spectra of modified surfaces contain additional lines of modified surface layer. In particular, the lines at 1882, 1785 and 1715 cm⁻¹ correspond to vibrations of oxygen containing groups which form on the PTFE surface after post-treatment oxidation in atmosphere. The line at 985 cm⁻¹ corresponds to vibrations of unsaturated C═C groups.

FIG. 22. shows the results of a quantitative analysis of the intensity of new groups appearing in the surface layer of the PTFE as a function of the ion fluence. The concentration of the oxygen containing groups and unsaturated groups clearly increases with the ion fluence.

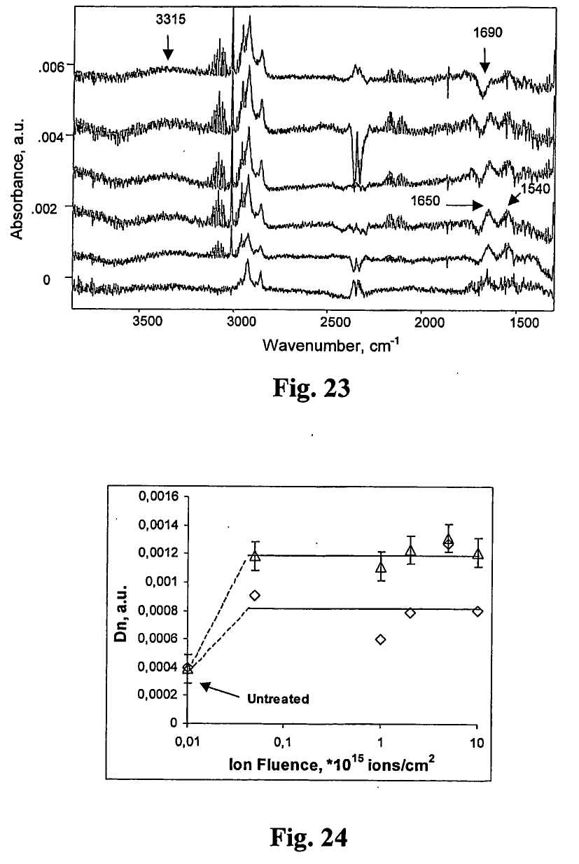

FIG. 23. contains FTIR ATR difference spectra of PIII modified PTFE before and after HRP protein attachment. After subtraction the spectra show only the lines corresponding to new groups which appear after soaking in the protein solution. The lines at 3315 cm-1 (Amide A), 1650 cm-1 (Amide I) and 1540 cm-1 (Amide II) are due to vibrations in protein molecules attached to PTFE surface. The intensity of protein lines appearing in the spectra taken from the untreated PTFE surface is close to the level of noise in the spectra while significant intensities are observed in spectra of the PIII modified PTFE surface.

The normalized absorbance for Amide A and Amide II lines as a function of ion fluence are shown in FIG. 24. According to the intensity of these lines, the concentration of attached HRP on the PTFE surface increases sharply after PIII treatment. However, the concentration does not depend strongly on the ion fluence. The fact that the concentration of attached protein does not increase with increased structural changes in the polymer indicates that the attached protein concentration saturates on PIII modified surface at relatively low fluence.

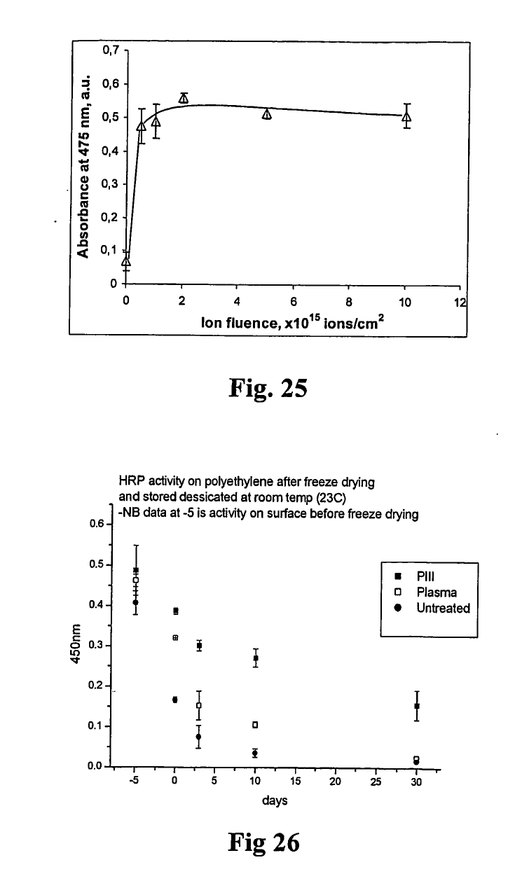

FIG. 25 contains the results of TMB test of active HRP protein on PTFE surface after PIII treatment. The high absorbance value of the TMB test for the PIII treated PTFE surface is similar to the maximal value observed for PIII modified polyethylene surfaces.

### Example 12

### Freeze Drying Horseradish Peroxidase on Polyethylene

### Material and Methods

Polyethylene surfaces were exposed to plasma or PIII plasma treatment according to the method outlined in Example 1. Both plasma and PIII plasma-treated and untreated surfaces were incubated overnight in horseradish peroxidase (50 ug/ml) in 10 mM phosphate buffer pH7. Next day the samples were washed in 10 mM phosphate buffer pH 7 six times, 20 minutes each time. To the last wash we added sucrose to a final concentration of 2.5%. The solution was then frozen with the samples in a 500 ml round bottom flask or in a 50 ml falcon tube by immersing the container in liquid nitrogen. When frozen the water was removed by attaching the round bottom flask to a Dynavac FD1 freeze dryer. Falcon tubes were placed inside the freeze dryer. We then freeze dried overnight. Freeze drying is a process in which the aqueous content of the materials is removed by sublimation into a vacuum. A successful freeze drying step will enable the function of the attached molecule to be restored upon rehydration. After freeze drying overnight the samples were removed and placed in a sealed container with desiccant and stored at 23 C. Samples were rehydrated and exposed to the HRP activity assay as in Example 1 at selected time points following freeze drying.

### Results and Discussion

The results shown in FIG. 26 demonstrate that significant activity of HRP bound to the polyethylene surface was retained for the surface that was exposed to PIII plasma-treatment even at the longest time point of 30 days. Furthermore, activity of the PIII plasma-treated surface was significantly higher than for both untreated and plasma only treated surfaces.

It is to be understood that the present invention has been described by way of example only and that modifications and/or alterations thereto, which would be apparent to a person skilled in the art based upon the disclosure herein, are also considered to fall within the scope and spirit of the invention, as defined in the appended claims.

### REFERENCES

1. S. Aibara, H. Yamashita, E. Mori, M. Kato, and Y. Morita, “Isolation and characterization of five neutral isoenzymes of horseradish peroxidase”, J Biochem (Tokyo), 1982 (92): 531-539.
2. G. Cohen, M. Kim and V. Oguwu, “A modified catalase assay suitable for a plate reader and for the analysis of brain cell cultures”, Journal of Neuroscience Methods, 1996, (67): 53-56.
3. Schena, M.; Shalon, D.; R. W. Davis; P. O. Brown Quantitative Monitoring of Gene Expression Patterns with a Complementary DNA Microarray Science 1995, 270, 467-470.
4. Kamal, J. K. A.; Behere, D. V. Thermal and conformational stability of seed coat soybean peroxidase Biochemistry 2002, 41, 9034-9042.
5. Wu W J, Vrhovski B, Weiss A S; J. Cell Biol. (1999) 274, 21719-21724
6. Stone P J, Morris S M, Griffin S, Mithieux S, Weiss A S; Am. J. Respir. Cell Mol. Biol. (2001) 22, 733-9.

1. An activated polymer substrate capable of binding a functional biological molecule, the substrate comprising a hydrophilic surface activated to enable binding to said biological molecule and a sub-surface comprising a plurality of cross-linked regions.

2. The activated polymer substrate according to claim 1 which comprises homopolymer, co-polymer, one or more polymer containing materials, mixtures or blends or a polymer matrix composite.

3. The activated polymer substrate according to claim 1 wherein the polymer substrate is a block, sheet, film, strand, fibre, piece or particle, powder, shaped article, woven fabric or massed fibre pressed into a sheet.

4. The activated polymer substrate according to claim 1 wherein the polymer substrate forms, or is a component of, a device selected from a diagnostic kit, a biosensor, a fuel cell or device for chemical processing, a cell or tissue culture scaffold, an analytical plate, an assay component or a medical device.

5. The activated polymer substrate according to claim 4 wherein the medical device is selected from a contact lens, a stent, a pace maker, a hearing aid, a prosthesis, an artificial joint, a bone or tissue replacement material, an artificial organ or artificial skin, an adhesive, a tissue sealant, a suture, staple, nail, screw, bolt or other device for surgical use or other implantable device.

6. The activated polymer substrate according to claim 1 wherein the polymer comprises one or more of polyolefins, blends of polyolefins with other polymers or rubbers; polyethers, polyamides, polyimides; polycarbonates; halogenated polymers, aromatic polymers, ketone polymers, methacrylate polymers and polyesters.

7. The activated polymer substrate according to claim 1 wherein the polymer comprises one or more of low density polyethylene (LDPE), polypropylene (PP), high density polyethylene (HDPE), ultra high molecular weight polyethylene (UHMWPE), polyoxymethylene (Acetal), poly(hexamethylene adipamide) (Nylon 66), polyvinylidenefluoride (PVDF), polytetra-fluoroethylene (PTFE), fluorinated ethylenepropylene copolymer (FEP), polyvinyl chloride (PVC), polystyrene (PS), polyetheretherketone (PEEK), polymethylmetl˜acrylate (PMMA), polyethylene terephthalate (PET), ABS and ethylene propylene diene mixture (EPDM).

8. A polymer substrate functionalised with a functional biological molecule, the functionalised polymer substrate comprising a hydrophilic surface with the biological molecule bound thereto and a sub-surface comprising a plurality of cross-linked regions.

9. The polymer substrate according to claim 8 which comprises homo-polymer, copolymer, one or more polymer containing materials, mixtures or blends or a polymer matrix composite.

10. The polymer substrate according to claim 8 wherein the polymer substrate is a block, sheet, film, strand, fibre, piece or particle, powder, shaped article, woven fabric or massed fibre pressed into a sheet.

11. The polymer substrate according to claim 8 wherein the polymer substrate forms, or is a component of, a device selected from a diagnostic kit, a biosensor, a fuel cell or device for chemical processing, a cell or tissue culture scaffold, an analytical plate, an assay component or a medical device.

12. The polymer substrate according to claim 11 wherein the medical device is selected from a contact lens, a stent, a pace maker, a hearing aid, a prosthesis, an artificial joint, a bone or tissue replacement material, an artificial organ or artificial skin, an adhesive, a tissue sealant, a suture, staple, nail, screw, bolt or other device for surgical use or other implantable device.

13. The polymer substrate according to claim 8 wherein the polymer comprises one or more of polyolefins, blends of polyolefins with other polymers or rubbers; polyethers, polyamides, polyimides; polycarbonates; halogenated polymers, aromatic polymers, ketone polymers, methacrylate polymers and polyesters.

14. The polymer substrate according to claim 8 wherein the polymer comprises one or more of low density polyethylene (LDPE), polypropylene (PP), high density polyethylene (HDPE), ultra high molecular weight polyethylene (UHMWPE), polyoxymethylene (Acetal), poly(hexamethylene adipamide) (Nylon 66), polyvinylidenefluoride (PVDF), polytetra-fluoroethylene (PTFE), fluorinated ethylenepropylene copolymer (FEP), polyvinyl chloride (PVC), polystyrene (PS), polyetheretherketone (PEEK), polymethylmethacrylate (PMMA), polyethylene terephthalate (PET), ABS and ethylene propylene diene mixture (EPDM).

15. The polymer substrate according to claim 8 wherein the biological molecule comprises one or more of amino acids, peptides, proteins, glycoproteins, lipoproteins, nucleotides, oligonucleotides, nucleic acids, lipids, carbohydrates.

16. The polymer substrate according to claim 8 wherein the biological molecule comprises a drug or drug target.

17. The polymer substrate according to claim 8 wherein the biological molecule comprises one or more of antibodies, immunoglobulins, receptors, enzymes, neurotransmitters, cytokines, hormones, complimentarity determining proteins, DNA, RNA and active fragments thereof.

18. The polymer substrate according to claim 8 wherein the biological molecule comprises one or more molecules that are integral to or attached to cells or cellular components.

19. The polymer substrate according to claim 8 wherein the biological molecule is covalently bound to the activated polymer.

20. A device comprising an activated polymer substrate capable of binding a functional biological molecule, the substrate comprising a hydrophilic surface activated to enable binding to said biological molecule and a sub-surface comprising a plurality of cross-linked regions.

21. The device according to claim 20 wherein the polymer substrate comprises homopolymer, co-polymer, one or more polymer containing materials, mixtures or blends or a polymer matrix composite.

22. The device according to claim 20 wherein the polymer substrate is a block, sheet, film, strand, fibre, piece or particle, powder, shaped article, woven fabric or massed fibre pressed into a sheet.

23. The device according to claim 20 which is a diagnostic kit, a biosensor, a fuel cell or device for chemical processing, a cell or tissue culture scaffold, an analytical plate, an assay component or a medical device.

24. The device according to claim 23 wherein the medical device is selected from a contact lens, a stent, a pace maker, a hearing aid, a prosthesis, an artificial joint, a bone or tissue replacement material, an artificial organ or artificial skin, an adhesive, a tissue sealant, a suture, staple, nail, screw, bolt or other device for surgical use or other implantable device.

25. The device according to claim 20 wherein the polymer comprises one or more of polyolefins, blends of polyolefins with other polymers or rubbers; polyethers, polyamides, polyimides; polycarbonates; halogenated polymers, aromatic polymers, ketone polymers, methacrylate polymers and polyesters.

26. The device according to claim 20 wherein the polymer comprises one or more of low density polyethylene (LDPE), polypropylene (PP), high density polyethylene (HDPE), ultra high molecular weight polyethylene (UHMWPE), polyoxymethylene (Acetal), poly(hexamethylene adipamide) (Nylon 66), polyvinylidenefluoride (PVDF), polytetra-fluoroethylene (PTFE), fluorinated ethylenepropylene copolymer (FEP), polyvinyl chloride (PVC), polystyrene (PS), polyetheretherketone (PEEK), polymethylmethacrylate (PMMA), polyethylene terephthalate (PET), ABS and ethylene propylene diene mixture (EPDM).

27. A device comprising a polymer substrate functionalised with a functional biological molecule, the functionalised polymer substrate comprising a hydrophilic surface with a biological molecule bound thereto and a sub-surface comprising a plurality of cross-linked regions.

28. The device according to claim 27 wherein the polymer substrate comprises homopolymer, co-polymer, one or more polymer containing materials, mixtures or blends or a polymer matrix composite.

29. The device according to claim 27 wherein the polymer substrate is a block, sheet, film, strand, fibre, piece or particle, powder, shaped article, woven fabric or massed fibre pressed into a sheet.

30. The device according to claim 27 which is a diagnostic kit, a biosensor, a fuel cell or device for chemical processing, a cell or tissue culture scaffold, an analytical plate, an assay component or a medical device.

31. The device according to claim 30 wherein the medical device is selected from a contact lens, a stent, a pace maker, a hearing aid, a prosthesis, an artificial joint, a bone or tissue replacement material, an artificial organ or artificial skin, an adhesive, a tissue sealant, a suture, staple, nail, screw, bolt or other device for surgical use or other implantable device.

32. The device according to claim 27 wherein the polymer comprises one or more of polyolefins, blends of polyolefins with other polymers or rubbers; polyethers, polyamides, polyimides; polycarbonates; halogenated polymers, aromatic polymers, ketone polymers, methacrylate polymers and polyesters.

33. The device according to claim 27 wherein the polymer comprises one or more of low density polyethylene (LDPE), polypropylene (PP), high density polyethylene (HDPE), ultra high molecular weight polyethylene (UHMWPE), polyoxymethylene (Acetal), poly(hexamethylene adipamide) (Nylon 66), polyvinylidenefluoride (PVDF), polytetra-fluoroethylene (PTFE), fluorinated ethylenepropylene copolymer (FEP), polyvinyl chloride (PVC), polystyrene (PS), polyetheretherketone (PEEK), polymethylmethacrylate (PMMA), polyethylene terephthalate (PET), ABS and ethylene propylene diene mixture (EPDM).

34. The device according to claim 27 wherein the biological molecule comprises one or more of amino acids, peptides, proteins, glycoproteins, lipoproteins, nucleotides, nucleic acids, lipids, carbohydrates.

35. The device according to claim 27 wherein the biological molecule comprises a drug or drug target.

36. The device according to claim 27 wherein the biological molecule comprises one or more of antibodies, immunoglobulins, receptors, enzymes, neurotransmitters, cytokines, hormones, complimentarity determining proteins, DNA, RNA and active fragments thereof.

37. The device according to claim 27 wherein the biological molecule comprises one or more molecules that are integral to or attached to cells or cellular components.

38. The device according to claim 27 wherein the biological molecule is covalently bound to the activated polymer.

39. A method of producing an activated polymer substrate comprising exposing a surface of a polymer substrate to plasma treatment with a suitable plasma forming gas, under plasma immersion ion implantation conditions.

40. A method of producing a polymer substrate functionalised with a biological molecule, comprising steps of:
(a) exposing a surface of a polymer substrate to plasma treatment with a suitable plasma forming gas, under plasma immersion ion implantation conditions;
(b) incubating the surface treated according to step (a) with a desired biological molecule.

41. An activated polymer substrate produced according to a method comprising exposing a surface of a polymer substrate to plasma treatment with a suitable plasma forming gas, under plasma immersion ion implantation conditions.

42. A polymer substrate functionalised with a biological molecule produced according to a method comprising steps of:
(a) exposing a surface of a polymer substrate to plasma treatment with a suitable plasma forming gas, under plasma immersion ion implantation conditions;
(b) incubating the surface treated according to step (a) with a desired biological molecule.

43. An activated polymer according to claim 1 which is freeze dried.
